(19) United States
(12) Patent Application Publication
(10) Pub. No.: US 2024/0100061 A1
(43) Pub. Date: Mar. 28, 2024

(54) Compositions of Iboga Alkaloids and Methods of Treatment

(71) Applicants: The Board of Trustees of the Leland Stanford Junior University, Stanford, CA (US); Soneira Inc., P.B.C., Incline Village, NV (US)

(72) Inventors: Nolan R. Williams, Half Moon Bay, CA (US); Ian Kratter, Stanford, CA (US); AnnaMarie Daniels, Incline Village, NV (US); Gregory Bird, Incline Village, NV (US)

(73) Assignees: The Board of Trustees of the Leland Stanford Junior University, Stanford, CA (US); Soneira Inc., P.B.C., Incline Village, NV (US)

(21) Appl. No.: 18/467,324

(22) Filed: Sep. 14, 2023

Related U.S. Application Data
(60) Provisional application No. 63/375,679, filed on Sep. 14, 2022.
(60) Provisional application No. 63/382,474, filed on Nov. 4, 2022.
(60) Provisional application No. 63/448,116, filed on Feb. 24, 2023.

(51) Int. Cl. A61K 31/55; A61K 9/00; A61K 33/06; A61P 9/06; A61P 25/24

(52) U.S. Cl. CPC A61K 31/55; A61K 9/0053; A61K 33/06; A61P 9/06; A61P 25/24

(57) ABSTRACT

Compositions comprising an iboga alkaloid and a cardioprotective agent are provided. Use of the compositions in treating neuropsychiatric disorders are described, where the cardioprotective agent is administered before, during and/or after the iboga alkaloid.

### CROSS-REFERENCE TO RELATED APPLICATIONS

This application claims the benefit of U.S. Provisional Application No. 63/375,679, filed Sep. 14, 2022, U.S. Provisional Application No. 63/382,474, filed Nov. 4, 2022, and U.S. Provisional Application No. 63/448,116, filed Feb. 24, 2023, each of which is incorporated herein by reference in its entirety.

### TECHNICAL FIELD

The subject matter described herein relates to compositions comprising an iboga alkaloid compound and a cardioprotective agent, and to methods of treatment using the same.

### BACKGROUND

Neuropsychiatric disorders encompass brain diseases or dysfunctions that cause a psychiatric symptom. Neuropsychiatric disorders include depression, schizophrenia, post-traumatic stress disorder, and anxiety disorders. They also include neurodegenerative diseases such as Alzheimer's disease, Parkinson's disease, Huntington's disease ataxia, Lewy body disease and others. Many of these neuropsychiatric disorders have few treatment options and there remains a need for treating the disorders and their symptoms.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In an embodiment, a method for treating a neuropsychiatric disorder of the brain is provided. The method comprises administering, or instructing to administer, to a subject a cardioprotective agent in an amount effective to achieve a physiologic effect to reduce risk of long QT syndrome and administering an iboga alkaloid or salt thereof.

In one embodiment, the physiologic effect is achieved when the cardioprotective agent is at a threshold concentration in the blood for a period of time, and the iboga alkaloid or salt thereof is administered during the period of time. In one embodiment, the period of time is at least about 1 hour, 2 hours, 4 hours, 6 hours, 8 hours, 10 hours or 12 hours, or is between about 1-12 hours, 1-10 hours, 1-8 hours, or 1-6 hours.

In one embodiment, the iboga alkaloid or salt thereof or a metabolite of the iboga alkaloid is at a therapeutic concentration in the blood for a treatment period, and the method further comprises administering to the subject a further amount of the cardioprotective agent to achieve a physiologic effect to reduce risk of a long QT syndrome.

In one embodiment, the further amount of the cardioprotective agent is administered parenterally or orally.

In one embodiment, the method further comprises assessing a baseline QT interval, or receiving information on a baseline QT interval, of the subject prior to administering the cardioprotective agent, after administering the cardioprotective agent, and/or after administering the iboga alkaloid or salt thereof.

In one embodiment, the cardioprotective agent is administered in an amount effective to achieve a physiologic effect to reduce risk of a drug-induced or acquired long QT syndrome.

In one embodiment, the iboga alkaloid or salt thereof is administered orally, parenterally or intrathecally.

In one embodiment, the iboga alkaloid or salt thereof is ibogaine or an analog of ibogaine.

In one embodiment, the iboga alkaloid or salt thereof is ibogaine hydrochloride.

In one embodiment, administering, or instructing to administer, the cardioprotective agent comprises orally administering or instructing to orally administer.

In one embodiment, the cardioprotective agent is selected from the group consisting of a mineral, a sodium channel blocker (a class 1B antiarrhythmic), a potassium channel blocker, an hERG (human ether-a-go-go-related gene) channel agonist, and beta adrenoceptor agonists.

In one embodiment, the sodium channel blocker is selected from the group consisting of mexiletine, tocainide, lidocaine, flecainide, and R-56865 (2-benzothiazolamine, N-(1-(4-(4-fluorophenoxy)butyl)-4-piperidinyl)-N-methyl).

In one embodiment, the potassium channel blocker is selected from the group consisting of amiodarone and ranolazine.

In one embodiment, the mineral is selected from the group consisting of magnesium, calcium and potassium.

In one embodiment, the mineral is in the form a salt, and wherein said administering or instructing to administer the cardioprotective agent comprises administering or instructing to administer prior to said administering the iboga alkaloid or salt thereof.

In one embodiment, the mineral is in the form a salt, and wherein said administering or instructing to administer the cardioprotective agent comprises administering or instructing to administer concurrent with and/or subsequent to said administering the iboga alkaloid or salt thereof.

In one embodiment, wherein the hERG channel agonist is selected from the group consisting of RPR260243 ([(3R,4R)-4-[3-(6-methoxy-quinolin-4-yl)-3-oxo-propyl]-1-[3-(2,3,5 trifluorophenyl)-prop-2-ynyl]-piperidine-3-carboxylic acid]), PD-118057 ([2-(4-[2-(3,4-dichloro-phenyl)-ethyl]-phenylamino)-benzoic acid]), and NS1643 (N,N′-bis[2-hydroxy-5-(trifluoromethyl)phenyl]-urea).

In one embodiment, the beta adrenoceptor agonist is isoproterenol.

In one embodiment, the cardioprotective agent is (i) not a CYP2D6 inhibitor or (ii) a CYP2D6 inhibitor administered at a dose ineffective to inhibit CYP2D6 or (iii) not amiodarone.

In one embodiment, the cardioprotective agent is a magnesium salt, and wherein said magnesium salt is administered in an amount of between about 50-8000 mg per day.

In one embodiment, the iboga alkaloid or salt thereof is a salt of ibogaine and wherein said administering the salt of ibogaine comprises administering between about 200-2500 mg or between about or 800-2400 mg.

In one embodiment, the neuropsychiatric disorder is traumatic brain injury. In one embodiment, the traumatic brain injury is mild, moderate or severe. In one embodiment, the subject exhibits a persistent symptom caused by the traumatic brain injury. In one embodiment, the traumatic brain injury is chronic. In one embodiment, the persistent symptom is selected from the group consisting of post-traumatic stress disorder, depression, anxiety or suicidal ideation. In one embodiment, the persistent symptom is selected from the group consisting of endocrine dysfunction, sleep disturbance, obstructive sleep apnea, chronic pain, orthopedic problems, headache, substance abuse, sexual health problems, cognitive impairment, and vestibular or vision impairment.

In one embodiment, the neuropsychiatric disorder is a neurological disorder or a neurodegenerative disease.

In one embodiment, the neurological disorder or the neurodegenerative disease is selected from the group consisting of Alzheimer's disease, mild cognitive impairment, Lewy-body dementia, progressive supranuclear palsy, Parkinson's disease, frontotemporal dementia, Tourette's syndrome, suspected chronic traumatic encephalopathy (CTE), multiple sclerosis, and amyotrophic lateral sclerosis.

In one embodiment, the neuropsychiatric disorder is selected from post-traumatic stress disorder and obsessive compulsive disorder.

In one embodiment, the subject exhibits a persistent symptom caused by the post-traumatic stress disorder or obsessive compulsive disorder.

In one embodiment, the persistent symptom is selected from the group consisting of depression, anxiety or suicidal ideation.

In one embodiment, the neuropsychiatric disorder is suspected chronic traumatic encephalopathy (CTE) and said treating is effective to slow progression of CTE.

In an embodiment, a method for treating a neuropsychiatric disorder of the brain in a subject is provided. The method comprises providing to the subject a cardioprotective agent and a unit dose of an iboga alkaloid or salt thereof, the cardioprotective agent provided in an amount sufficient for a plurality of doses; instructing the subject to self-administer one or more doses of the cardioprotective agent; instructing to administer, or administering, to the subject while under clinical or medical supervision the unit dose of the iboga alkaloid or salt thereof, and optionally administering to the subject while under clinical or medical supervision for administration of the unit dose of ibogaine, a further dose of the cardioprotective agent; and optionally instructing the subject to self-administer one or more doses of the cardioprotective agent subsequent to the administration of the unit dose of the iboga alkaloid or salt thereof.

In one embodiment, the cardioprotective agent is provided to the subject as a plurality of unit doses of the cardioprotective agent.

In one embodiment, the cardioprotective agent is administered in an amount effective to achieve a physiologic effect to reduce risk of long QT syndrome.

In one embodiment, the physiologic effect is achieved when the cardioprotective agent is at a threshold concentration in the blood for a period of time, and the unit dose of iboga alkaloid or salt thereof is administered during the period of time.

In one embodiment, the unit dose of iboga alkaloid or salt thereof or a metabolite of the iboga alkaloid provides a therapeutic concentration in the blood for a treatment period, and the method further comprises administering to the subject a further amount of the cardioprotective agent during the treatment period.

In one embodiment, the further amount of the cardioprotective agent is administered parenterally or orally.

In one embodiment, the method further comprises assessing a baseline QT interval, or receiving information on a baseline QT interval, of the subject.

In one embodiment, the cardioprotective agent is administered in an amount effective to achieve a physiologic effect to reduce risk of a drug-induced or acquired long QT syndrome.

In one embodiment, a method for treating a neuropsychiatric disorder of the brain in a subject is provided. The method comprises providing a device comprising a plurality of unit doses of a cardioprotective agent and a unit dose of an iboga alkaloid or salt thereof; instructing a subject to self-administer a first portion of the plurality of unit doses of the cardioprotective agent; instructing to administer or administering under clinical or medical supervision the unit dose of the iboga alkaloid or salt thereof to the subject; and administering or instructing the subject to self-administer a second portion of the plurality of unit doses of the cardioprotective agent.

In one embodiment, the administering or instructing the subject to self-administer the second portion comprises administering or instructing the subject to self-administer while under clinical or medical supervision for the unit dose of iboga alkaloid or salt thereof.

In one embodiment, the method further comprises administering or instructing the subject to self-administer a third portion of the plurality of unit doses of the cardioprotective agent after the second portion of the unit doses of the cardioprotective agent is administered.

In one embodiment, the instructing the subject to self-administer comprises instructing the subject to self-administer a unit dose of the cardioprotective agent once daily for a period of about 2, 3, 4, or 5 days prior to the unit dose of the iboga alkaloid or salt thereof being administered.

In one embodiment, the device further comprise a second unit dose of the iboga alkaloid or salt thereof, the second unit dose being less than the unit dose of the iboga alkaloid or salt thereof.

In one embodiment, the device is a blister pack comprising each unit dose in a separate blister of the blister pack.

In an embodiment, a composition comprises an orally administrable dosage form comprising an iboga alkaloid or salt thereof and a salt of magnesium, potassium or calcium, the salt of magnesium, potassium or calcium present in the dosage form in an amount effective to achieve a physiologic effect to reduce risk of a drug-induced long QT syndrome.

In one embodiment, the dosage form is a solid or a liquid. In one embodiment, the solid dosage form is for oral ingestion. In one embodiment, the solid dosage form is selected from the group consisting of a sublingual composition, a buccal composition, a powder, a capsule, a pill, and a tablet.

In one embodiment, the salt of magnesium, potassium or calcium is formulated for immediate release upon oral administration of the dosage form.

In one embodiment, the iboga alkaloid or salt thereof is formulated for delayed release after oral administration of the dosage form.

In one embodiment, iboga alkaloid or salt thereof is formulated for release at least 2 hours after the salt of magnesium, potassium or calcium is released from the dosage form.

In an embodiment, a kit comprises a device comprising a plurality of unit doses of a cardioprotective agent and a unit dose of an iboga alkaloid or salt thereof, and instructions for use.

In one embodiment, one or more of the unit doses of cardioprotective agent in the plurality comprise an oral dosage form.

In one embodiment, the oral dosage form is a powder, and the instructions for use instruct to prepare a solution for ingestion.

In one embodiment, the unit dose of cardioprotective agent is in the form of a liquid selected from the group consisting of an elixir, a syrup, a suspension and a solution.

In one embodiment, the liquid is formulated for parenteral administration or for nasal administration.

In one embodiment, the unit dose of iboga alkaloid or salt thereof is a solid or a liquid. In one embodiment, the unit dose is a solid selected from the group consisting of a capsule, a pill, a powder and a tablet.

In one embodiment, the unit dose is a liquid selected from the group consisting of an elixir, a syrup, a suspension and a solution.

In one embodiment, the unit dose is a liquid.

In one embodiment, the liquid is formulated for parenteral administration or for nasal administration.

In one embodiment, the unit dose is formulated for oral administration as a liquid selected from the group consisting of an elixir, a syrup, a suspension and a solution.

In one embodiment, the unit dose of ibogaine is a unit dosage form of the composition as described herein.

In one embodiment, the device is a blister pack comprising each unit dose in a separate blister of the blister pack.

In one embodiment, the cardioprotective agent is an electrolyte.

In one embodiment, the electrolyte is magnesium, potassium or calcium.

In one embodiment, the electrolyte is a salt form of the electrolyte.

In one embodiment, the electrolyte is a salt form of magnesium selected from the group consisting of magnesium aspartate, magnesium bisglycenate, magnesium carbonate, magnesium chloride, magnesium citrate, magnesium gluconate, magnesium glycinate, magnesium hydroxide, magnesium malate, magnesium oxide, magnesium sulfate, and magnesium taurate.

In one embodiment, the cardioprotective agent is a magnesium salt for administration to the subject of at a dose of between about 50-8000 mg.

Treatments described herein utilize ibogaine and/or other biosimilar iboga alkaloids to treat conditions not previously understood to be safely treatable using these drugs. Also, as described infra, the dangerous cardiovascular side effects can be avoided by co-treatment using magnesium ions, calcium ions, and/or potassium ions. Dosing of patients using one or a combination of these ions can stabilize hERG potassium channels and prevent torsades de pointes. In many embodiments, prior to treatment using ibogaine or a similar iboga alkaloid, electrolyte levels in the patient are stabilized. Subsequently, the patient is dosed with the positive ions along with the delivery of ibogaine (or similar iboga alkaloid) which mitigates and/or prevents the deleterious cardiovascular side effects. This co-administration of compounds enables a significantly safer administration of ibogaine, enabling patients to benefit from the treatment effects discussed below.

In an embodiment, ibogaine's cardiotoxicity can be sufficiently negated by delivering ibogaine (or a cardiotoxic ibogaine analog or any salt thereof) in combination with any of: lidocaine, mexiletine, R-56865, flecanide, amiodarone, and ranolazine. In particular, mexiletine is well tolerated and considered safe for oral use. In an embodiment, a combination pill having a ratio of 4 mg mexiletine to 18 mg ibogaine is provided. However, in practice these ratios may be shifted from anywhere between 2-6 mg mexiletine to 15-20 mg ibogaine. Further, the total mass per dose can be increased in order to comfortably dose patients at a ratio of approximately 4 mg/kg mexiletine to 15-20 mg/kg ibogaine. In an embodiment, the resulting combination drug of mexiletine/ibogaine can be administered orally for convenience, and further to ensure that no raw ibogaine is packaged and sent to the public facing medical system. The combination drug can be used to treat addiction as well as any number of different brain conditions such as, but not limited to post-traumatic stress disorder, traumatic brain injury, depression, insomnia, Parkinson's disease, chronic traumatic encephalopathy, and/or any other condition for which ibogaine provides therapeutic effect. In an embodiment, a pharmaceutical composition comprising mexiletine or a salt thereof and ibogaine or a salt thereof, in the ratio of 2-6 mg mexiletine to 15-20 mg ibogaine is provided. In another embodiment, the composition is prepared to be orally administered. In another embodiment, a pharmaceutical composition comprising ibogaine or a salt thereof, in combination with any of: lidocaine, mexiletine, R-56865, flecanide, amiodarone, ranolazine, or any salt thereof is provided.

Provided are methods and compositions where an iboga alkaloid, such as ibogaine, repairs brain tissue which can be used to treat neurodegenerative diseases. Furthermore, empirical verification that brain age can be reversed via ibogaine treatment is provided. In embodiments, an magnetic resonance imaging (MRI) scan is conducted prior to treatment and/or after treatment, such as 1-7 days post treatment or 1-month post treatment, in order to confirm regrowth and therefore efficacy of the treatment. Brain age algorithms can be used as empirical verification of treatment.

In an aspect, a treatment of a neurodegenerative disease is provided. The treatment comprises stabilizing a patient's electrolyte level, administering to the patient a therapeutically effective amount of ibogaine, ibogaine derivative, or a pharmaceutically acceptable salt and/or solvate thereof, and co-administering a human ether-a-go-go related gene (hERG) stabilizing amount of magnesium ion, calcium ion, or potassium ion.

In an embodiment, the neurodegenerative disease is Alzheimer's disease, mild cognitive impairment, Lewy-body dementia, progressive supranuclear palsy, Parkinson's disease, or frontotemporal dementia. In an embodiment, the neuropsychiatric disease is one that is not a neurodegenerative disease, such as depression, obsessive compulsive disorder, Tourette's syndrome, post-traumatic stress disorder and traumatic brain injury.

In addition to the exemplary aspects and embodiments described above, further aspects and embodiments will become apparent by reference to the drawings and by study of the following descriptions.

Additional embodiments of the present methods and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE DRAWINGS

FIG. 1A is a bar graph showing combined results for Delis-Kaplan Executive Function System (D-KEFS) testing of inhibition score for the enrolled subjects at baseline, at immediate (4 days) post treatment and one month post treatment.

FIGS. 1B-1C are graphs showing individual results of D-KEFS testing of cognitive inhibition.

FIGS. 2A-2B are bar graphs showing results from assessment of working memory (FIG. 2A) and processing speed (FIG. 2B) using the Wechsler Adult Intelligence Scale 4th Edition (WAIS-IV) test at baseline, at immediate (4 days) post treatment and one month post treatment.

FIGS. 3A-3B are graphs showing post-traumatic stress disorder (PTSD) symptom severity as measured using Clinician-Administered PTSD Scale for DSM-5 (CAPS-5), collectively for the enrolled subjects (FIG. 3A) and individually (FIG. 3B), at baseline, at immediate (4 days) post treatment and one month post treatment.

FIGS. 4A-4B are graphs showing depression symptom severity as measured using Montgomery-Asberg Depression Rating Scale (MADRS), collectively for the enrolled subjects (FIG. 4A) and individually (FIG. 4B), at baseline, immediately after (4 days) treatment, and one month post treatment.

FIGS. 5A-5B are graphs showing anxiety symptom severity as measured using Hamilton-Anxiety Rating Scale (HAM-A), collectively for the enrolled subjects (FIG. 5A) and individually (FIG. 5B), at baseline, immediately after (4 days) treatment, and one month post treatment.

FIGS. 6A-6B are graphs showing results from the Moral Injury Symptom Scale (MISS), collectively for the enrolled subjects (FIG. 6A) and individually (FIG. 6B), at baseline and one month post treatment.

FIG. 7A is a graph showing results from the World Health Organization Disability Assessment Scale 2.0 (WHODAS 2.0) assessment for each subject at baseline (prior to ibogaine treatment), about one week after treatment, and one month after treatment; the dashed line represents the means of the individual scores.

FIG. 7B is a bar graph of self-reported disability using the WHODAS 2.0 assessment showing the combined scores at baseline and one-month after treatment of the enrolled subjects.

FIG. 8 is a bar graph of the percent reduction in PTSD symptoms from baseline to one-month post treatment assessed using CAPS-5 for the subjects reporting an alcohol use disorder and a non-alcohol use disorder, after treatment with ibogaine.

FIGS. 9A-9E are graphs showing results of neuropsychological testing, where baseline scores and scores one week post and one month post treatment are shown in percentile relative to age peers in detection, reaction time and sustained attention (FIG. 9A, where lower scores for detection represent improvement; verbal memory and visuospatial memory (FIG. 9B), processing speed (FIG. 9C); cognitive inhibition, cognitive flexibility composite, phenomic fluency, working memory, problem solving (collectively, Executive Function, FIG. 9D); and semantic fluency (language; FIG. 9E), where Y axis represents percentile; X represents mean; middle line represents median; Whisker lines represent interquartile range (IQR); single dots represent participants with a score>±1.5 IQR. *p(FDR)<0.05; **p(FDR)<0.01; ***p(FDR)<0.001.

FIGS. 10A-10C show a correlation matrix of functional connectivity network changes in regions of interest in the brain, as measured using functional MRI, in subjects at baseline, one week after treatment and one month after treatment.

FIG. 11 is a graph showing changes in functional connectivity Z-scores for significant functional connectivity networks subjects at baseline, one week after treatment with ibogaine and one month after treatment.

FIGS. 12A-12B are graphs showing changes in functional connectivity Z-scores for significant functional connectivity networks in subjects at baseline, one week after treatment with ibogaine and one month after treatment, with FIG. 12B offering a detail of the change in functional connectivity of the right hemisphere (RH) central executive network (CEN) in the precuneus brain region to the left hemisphere (LH) default mode network (DMN) in the parietal brain region.

FIGS. 13A-13C provide visual representations of the reorganization of certain functional connectivity pairs in the brain network in subjects at baseline (FIG. 13A), one week after treatment (FIG. 13B), and one month after treatment (FIG. 13C).

FIG. 14 is a graph showing group wide distribution of the functional connectivity of the right hemisphere (RH) central executive network (CEN) in the precuneus brain region to the left hemisphere (LH) default mode network (DMN) in the parietal brain region in subjects at baseline (v2), one week after treatment with ibogaine (v3) and one month after treatment (v4).

FIG. 15 is a graph showing changes in individual subjects of the functional connectivity of the right hemisphere (RH) central executive network (CEN) in the precuneus brain region to the left hemisphere (LH) default mode network (DMN) in the parietal brain region in subjects at baseline (v2) and one week after treatment (v3).

FIGS. 16A-16J are graphs showing correlation showing correlation between functional connectivity of the right hemisphere (RH) central executive network (CEN) in the precuneus brain region and the left hemisphere (LH) default mode network (DMN) in the parietal brain region and the clinical measure indicated on the x-axis of each graph. Values of the clinical measure for baseline are shown (open circles, solid line) and the change in functional connectivity (“delta FC”) from baseline and 1-week post treatment (squares, dotted line) and 1-month post treatment (triangles, dashed line) are shown for: depression symptom severity as measured using MADRS (FIG. 16A), PTSD symptoms assessed using CAPS-5 (FIG. 16B); in subjects with PTSD symptoms (1sPTSD) assessed using CAPS-5 (FIG. 16C), and WHODAS assessments of cognition (FIG. 16D), cognition average (FIG. 16E), mobility (FIG. 16F), mobility average (FIG. 16G), self-care (FIG. 16H), self-care average (FIG. 16I) and getting along (FIG. 16J).

FIGS. 16K-16N are graphs showing correlations of functional connectivity of right hemisphere CEN to left hemisphere DMN at one month post treatment from baseline and change in clinical scores for depression (FIG. 16K), PTSD (FIG. 16L), problem solving (FIG. 16M) and cognitive inhibition (FIG. 16N).

FIG. 17A is a graph of cerebral white matter volume, in mm³, one month after treatment as a function of cerebral white matter volume, in mm³×10⁵, at baseline (before treatment).

FIG. 17B is a graph of the change in cerebral white matter volume, in mm³, taken as the difference in cerebral white matter volume one month after treatment and at baseline, as a function of cerebral white matter mean volume, in mm³×10⁵, at baseline (before treatment) and one month after treatment.

FIG. 18A is a bar graph showing the effect size of a change in cerebral white matter volume in 47 brain regions as indicated along the x-axis, one month after treatment.

FIG. 18B is a bar graph showing the effect size of a cortical thickness change from baseline to one month after treatment for the brain regions indicated along the x-axis.

FIG. 19A shows the difference in predicted brain age from baseline, in years, for the subjects in the study of Example 1 at baseline, 4 days after treatment and one month after treatment.

FIG. 19B shows the individual predicted brain age trajectories, normalized to baseline, in years, at baseline (v2), 4 days after treatment (v3) and one month after treatment (v4).

FIG. 19C is a bar graph showing algorithm brain age, in years, for 24 of the subjects in the study of Example 1 prior to ibogaine treatment (baseline), one week after treatment and one month after treatment.

FIG. 19D is a graph at baseline, and 1-week and 1-month after treatment with ibogaine and a cardioprotective agent (open circles, solid line) and 1-week and 1-month after treatment with a transcranial magnetic stimulation technique, accelerated intermittent theta burst stimulation (aiTBS, solid circles, dashed line) of brain age estimate (difference from baseline), in years, for study participants.

FIG. 20A is a graph of score on the Delis-Kaplan Executive Function System (DKFS) Category Switching Task for individuals one month after treatment as a function of predicted brain age, in years, one month after treatment.

FIG. 20B is a graph of the difference between time per move on the Delis-Kaplan Executive Function System Tower Test between one month after treatment and baseline, as a function of difference between predicted brain age one month after treatment and baseline, in years.

FIG. 20C is a graph of difference in predicted brain age one month after treatment relative to baseline, in years, in subcortical (thalamus, caudate, putamen, hippocampus, amygdala, accumbens and ventral DC) grey matter volume one month after treatment relative to baseline.

FIG. 20D is a graph of difference in predicted brain age one month after treatment relative to baseline, in years, in brain stem volume one month after treatment relative to baseline.

FIG. 21A is a graph showing the correlation between change in relative cerebral blood perfusion (rCBF) and change in score on World Health Organization Disability Assessment Scale 2.0 (WHODAS 2.0) in the right anterior cingulate gyrus [MINI coordinates 12, 40, 18] from baseline to one-month post treatment.

FIG. 21B is a graph showing the correlation between change in cerebral blood flow and change in score on the Clinician-administered PTSD Scale for DSM-5 (CAPS-5) in the right middle cingulate gyrus [MINI coordinates 6, 8, 38], the change from baseline to one-month post treatment.

FIG. 21C is a graph showing the correlation between change in relative cerebral blood perfusion (rCBF) and change in score on the Montgomery-Asberg Depression Rating Scale (MADRS) in the dorsomedial prefrontal cortex (dmPFC) region within the right superior frontal gyrus [MINI coordinates 4, 42, 34], the change from baseline to one-month post treatment.

FIG. 21D is a graph showing the correlation between change in relative cerebral blood perfusion (rCBF) and change in score on WHODAS 2.0 in the right insula [MINI coordinates 42, 4, −8], from baseline to one-month post treatment.

FIG. 21E is a graph showing the correlation between change in relative cerebral blood perfusion (rCBF) and change in score on WHODAS 2.0 in the right planum polare [MINI coordinates 46, 4, −10], from baseline to one-month post treatment.

FIG. 21F is a graph showing the correlation between change in relative cerebral blood perfusion (rCBF) and change in score on WHODAS 2.0 in the right posterior orbital gyrus [MINI coordinates 34, 20, −14], from baseline to one-month post treatment.

FIG. 21G shows the change in relative cerebral blood flow in the right middle cingulate gyrus one month after treatment as a function of absolute improvement in PTSD symptoms using the CAPS assessment.

FIG. 21H shows the change in relative cerebral blood flow in the dorsomedial prefrontal cortex brain region one month after treatment as a function of absolute improvement in disability symptoms using the WHODAS assessment.

FIG. 21I shows the change in relative cerebral blood flow in the right anterior insula one month after treatment as a function of absolute improvement in disability symptoms using the WHODAS assessment.

FIG. 21J shows the change in relative cerebral blood flow in the dorsomedial prefrontal cortex brain region one month after treatment as a function of absolute improvement in disability symptoms using the MADRAS assessment.

FIG. 22 is a cross-sectional diagram of the brain showing regions of interest.

FIGS. 23A-23B are bar graphs showing the relative cerebral brain perfusion change from baseline to four days after treatment (FIG. 23A) or from baseline to one month after treatment (FIG. 23B), measured using Fisher's Z (Spearman rho), for the indicated PET map receptors abbreviated as follows: 5-HT1a (serotonin 5-hydroxytryptamine receptor subtype 1a), 5-HT1b (5-HT subtype 1b), 5-HT2a (5-HT subtype 2a), 5-HT4 (5-HT subtype 4), CB1 (cannabinoid receptor 1), D1 (dopamine D1), D2 (dopamine D2), DAT (dopamine transporter), F-DOPA (dopamine synthesis capacity), GABAa (gamma-aminobutric acid), Mu (Mu opioid receptor), SERT (serotonin transporter), vesicular acetylcholine transporter (VAChT), and mGluR5 (metabotropic glutamate receptor subtype 5).

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can comprise, consist essentially of, or consist of, the components disclosed.

All percentages, parts and ratios are based upon the total weight of the composition and all measurements made are at about 25° C., unless otherwise specified.

“Administration” refers to introducing an agent, such as an iboga alkaloid, into a subject or patient. Typically, an effective amount is administered, which amount can be determined by the treating physician or the like. Any route of administration, such as oral, topical, subcutaneous, peritoneal, intra-arterial, inhalation, vaginal, rectal, nasal, introduction into the cerebrospinal fluid, or instillation into body compartments can be used. The agent, such as an iboga alkaloid, may be administered by direct blood stream delivery, e.g. sublingual, buccal, intranasal, or intrapulmonary administration.

The related terms and phrases “administering” and “administration of’, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient.

“Ibogaine” refers to the compound:

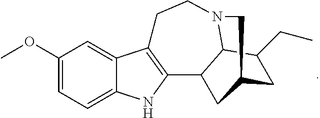

as well as pharmaceutically acceptable salts and pharmaceutically acceptable solvates thereof. It should be understood that where “ibogaine” is mentioned herein, one or more polymorphs of ibogaine can be utilized and are contemplated. Ibogaine is isolated from Tabernanth iboga, a shrub of West Africa. Reference to a derivative of ibogaine or an ibogaine derivative intends a compound other than ibogaine and based on the molecular core of ibogaine, and non-limiting examples of ibogaine derivatives are provided, for example, in WO2017/184531, which are incorporated by reference herein.

“Noribogaine” refers to the compound:

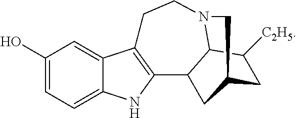

as well as pharmaceutically acceptable salts thereof or solvates thereof. Noribogaine can be prepared by demethylation of naturally occurring ibogaine. Demethylation may be accomplished by conventional techniques such as by reaction with boron tribromide/methylene chloride at room temperature followed by conventional purification. See, for example, Huffman, et al., J. Org. Chem 50:1460 (1985). Noribogaine can be synthesized as described, for example, in U.S. Patent Pub. Nos. 2013/0165647, 2013/0303756, and 2012/0253037, PCT Patent Publication No. WO 2013/040471 (includes description of making noribogaine polymorphs), and U.S. Pat. No. 9,617,274, each of which is incorporated herein by reference in its entirety.

“Neuropsychiatric” is used herein with reference to disorders of affect, cognition, and/or behavior that arise from a disorder in cerebral structure and/or function and/or from indirect effects of an extracerebral disease. Neuropsychiatric disorders include neurological disorders and neurodegenerative disorders.

As used herein, the term “patient” or “subject” refers to mammals and includes humans and non-human mammals.

The phrase “pharmaceutically acceptable” is employed herein to refer to those compounds, salts, compositions, dosage forms, etc., which are within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

“Pharmaceutically acceptable salt” refers to salts, including pharmaceutically acceptable partial salts, of a compound, which salts are derived from a variety of organic and inorganic counter ions well known in the art and include, by way of example only, hydrochloric acid, hydrobromic acid, phosphoric acid, sulfuric acid, methane sulfonic acid, phosphorous acid, nitric acid, perchloric acid, acetic acid, tartaric acid, lactic acid, succinic acid, citric acid, malic acid, maleic acid, aconitic acid, salicylic acid, thalic acid, embonic acid, enanthic acid, oxalic acid and the like, and when the molecule contains an acidic functionality, include, by way of example only, sodium, potassium, calcium, magnesium, ammonium, tetraalkylammonium, and the like.

“Therapeutically effective amount” or “therapeutic amount” refers to an amount of a drug or an agent that, when administered to a patient suffering from a condition, will have the intended therapeutic effect, e.g., alleviation, amelioration, palliation or elimination of one or more manifestations of the condition in the patient. The therapeutically effective amount may vary depending upon the patient and the condition being treated, the gender, weight and age of the subject, the severity of the condition, presence of co-morbidities, the salt, solvate, or derivative of the active drug portion chosen, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of an agent, in the context of treating a neurodegenerative disease or a movement disorder and/or symptoms thereof, refers to an amount of the agent that attenuates the disease or disorder; attenuates, reverses, or reduces the severity of a symptom or symptoms thereof; and/or prevents, delays, or reduces the severity of progression of the disease or disorder.

A “therapeutic level” of a drug is an amount of iboga alkaloid or pharmaceutically acceptable salt or solvate thereof that is sufficient to treat or prevent the disease or disorder and/or symptoms thereof, but not high enough to pose any significant risk to the patient. Therapeutic levels of drugs can be determined by tests that measure the actual concentration of the compound in the blood of the patient. This concentration is referred to as the “serum concentration.”

The term “treating” is used herein, for instance, in reference to methods of treating a neuropsychiatric disorder, and generally includes the administration of a compound or composition which reduces the frequency of, or delays the onset of, symptoms of a medical condition (e.g., a neuropsychiatric disorder or a neurological disorder or a neurodegenerative disorder) in a subject relative to a subject not receiving the compound or composition. This can include reversing, reducing, or arresting the symptoms, clinical signs, biological signed, and/or underlying pathology of a condition in a manner to improve or stabilize a subject's condition.

The term “unit dose” refers to a dose of drug provided to a patient to provide a therapeutic result, independent of the weight of the patient. The unit dose can be a standard form (e.g., a tablet or capsule). The unit dose may be administered as a single dose or a series of subdoses that collectively equal the single dose.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Iboga Alkaloid Compositions

Iboga alkaloids are alkaloid constituents of the Tabernathe iboga. Ibogaine, ibogaline, ibogamine and tabernathine are representative molecules, and are psychoactive. Despite being known for over a century, the full scope of effects on the human body have remained unclear. For example, ibogaine has been reported to be effective in treating substance addiction, such as alcohol addiction and drug addiction, including opioid and stimulant drugs, but has remained a less used pharmaceutical option due to its hallucinogenic (oneirogenic) effect and potential for neurotoxic and cardiovascular side effects. Approximately 1 in 300 people may suffer from cardiac arrest when treated with ibogaine. Given the dangers of the drug, ibogaine has been classified in the United States as a Schedule I controlled substance.

An exemplary iboga alkaloid is ibogaine. Ibogaine is a naturally occurring psychoactive molecule found in plants in the family Apocynaceae, most familiarly in Tabernanthe iboga. Ibogaine when orally administered to humans has a half-life of about 7.5 hours, and possibly longer in subjects that are poor CYP2D6 metabolizers. Ibogaine can cause long QT syndrome by blocking hERG potassium channels in the heart, leading to death. Ibogaine's cardiotoxicity renders it unsafe for use in patient treatment. The compositions and methods described herein provide a solution. Another exemplary iboga alkaloid is noribogaine, which has half-life of 28-49 hours. These compounds are lipophilic and distribute widely in the body and accumulate in fatty tissues, such as brain, heart and adipose.

Compositions comprising an iboga alkaloid or salt thereof and a cardioprotective agent are provided. The compositions are not limited to any particular chemical form of iboga alkaloid, and the compound may be present in the composition as a free base or as a pharmaceutically acceptable salt. The cardioprotective agent is, in an embodiment, a compound with activity to reduce risk of a drug-induced long QT syndrome and/or with activity to mitigate risk associated with drug-induced long QT syndrome. In other embodiments, the cardioprotective agent is a compound with activity to reduce long QT syndrome or QT prolongation in a subject. In other embodiments, the cardioprotective agent has a stabilization effect on cardiac membrane that is independent of QT changes.

Ibogaine and its principal metabolite noribogaine demonstrate moderate-to-weak affinity for a number of neurotransmitter receptors including N-methyl-D-aspartate, kappa and mu opioid, sigma-1 and −2, nicotinic acetylcholine, serotonin transporter, and dopamine transporter, among others (Litjens, R. P. W. et al., Clin. Toxicol., 54, 297-302 (2016); Corkery, J. M., Progress in Brain Research, 242.217-257 (Elsevier, 2018); Wasko, M. J. et al., ACS Chem. Neurosci. 9, 2475-2483 (2018)). Ibogaine also increases the transcription of neurotrophic factors including brain-derived neurotrophic factor (BDNF) and glial cell line-derived neurotrophic factor (GDNF) (Marton, S. et al., Front. Pharmacol. 10, (2019)) and increases cortical neuron dendritic arbor complexity in vitro (Cameron, L. P. et al., Nature, 589, 474-479 (2021)).

The iboga alkaloid may be present in the composition as a free base or as a salt, and in an embodiment, as a pharmaceutically acceptable acid addition salt. In an embodiment, the iboga alkaloid is a hydrochloride salt, exemplified by ibogaine hydrochloride, however other salts derived from organic or inorganic acids may also be used. Examples of such acids include, without limitation, those described above as pharmaceutically acceptable salts and the like.

In embodiments, the cardioprotective agent is a mineral, a sodium channel blocker (a class 1B antiarrhythmic), a potassium channel blocker, an hERG (human ether-a-go-go-related gene) channel agonist, and/or a beta adrenoceptor agonist. Exemplary sodium channel blockers are mexiletine, tocainide, lidocaine, flecainide, and R-56865 (2-benzothiazolamine, N-(1-(4-(4-fluorophenoxy)butyl)-4-piperidinyl)-N-methyl). Exemplary potassium channel blockers are amiodarone and ranolazine. Exemplary minerals are magnesium, calcium and/or potassium. Exemplary hERG channel agonists include RPR260243 ([(3R,4R)-4-[3-(6-methoxy-quinolin-4-yl)-3-oxo-propyl]-1-[3-(2,3,5 trifluorophenyl)-prop-2-ynyl]-piperidine-3-carboxylic acid]), PD-118057 ([2-(4-[2-(3,4-dichloro-phenyl)-ethyl]-phenylamino)-benzoic acid]), and NS1643 (N,N′-bis[2-hydroxy-5-(trifluoromethyl)phenyl]-urea). An exemplary beta adrenoceptor agonist is isoproterenol. The cardioprotective agent can be in salt, base or elemental form.

In an embodiment, the cardioprotective agent is not a CYP2D6 inhibitor, a CYP2D6 inhibitor administered at a dose ineffective to inhibit CYP2D6, and/or amiodarone.

In an embodiment, the cardioprotective agent is a mineral, and in an embodiment, the mineral is present in the composition in the form a salt. In an embodiment, the mineral salt is an electrolyte. In an embodiment, the electrolyte is calcium, sodium, potassium, phosphate, magnesium and/or chloride. Exemplary salts of magnesium include magnesium aspartate, magnesium aspartate hydrochloride, magnesium bisglycenate, magnesium carbonate, magnesium chloride, magnesium citrate, magnesium gluconate, magnesium glycinate, magnesium hydroxide, magnesium lactate, magnesium malate, magnesium oxide, magnesium sulfate, and magnesium taurate. Exemplary salts of potassium and calcium include potassium chloride, potassium citrate, potassium bicarbonate, calcium gluconate, calcium chloride, calcium citrate, calcium glubionate, calcium gluceptate, calcium carbonate, and calcium acetate.

In an embodiment, the salt form of the mineral or electrolyte is not a fatty acid salt form of the mineral or electrolyte. In an embodiment, a fatty acid refers to a carboxylic acid with a saturated or unsaturated, branched or linear, aliphatic chain. In an embodiment, the aliphatic chain has about four or more, five or more, six or more carbon atoms. For example, the aliphatic chain may have between about 4-26, 5-26, 6-26, 7-26 or 8-26 carbon atoms. In an embodiment, the cardioprotective agent is not a magnesium salt of a fatty acid. In an embodiment, the cardioprotective agent is not magnesium stearate.

In an embodiment, the cardioprotective agent is a magnesium salt that ionizes in an aqueous medium to a magnesium cation and an anion or anion pair, the anion or the anion pair having a molecular mass of less than about 200 g/mol, 225 g/mol, 250 g/mol, 275 g/mol or 280 g/mol. In an embodiment, the cardioprotective agent is a magnesium salt that is essentially completely soluble in water at 25° C. In an embodiment, the cardioprotective agent is a magnesium salt that has a water solubility at 25° C. of greater than about 5 g/L, 10 g/L, 25 g/L or 50 g/L.

In embodiments, the cardioprotective agent is in the composition in an amount effective to achieve a physiologic effect to reduce risk of a drug-induced long QT syndrome. For example, in embodiments where the cardioprotective agent is magnesium, potassium or calcium, the composition can include a salt of magnesium, potassium or calcium, where the salt form is present in the composition in an amount effective to achieve a physiologic effect on the QT interval. In embodiments, the cardioprotective agent is administered in an amount effective to shorten the QT interval and/or to reduce risk of developing drug-induced QT prolongation. In an embodiment, the effective amount is an amount to reduce risk associated with a drug-induced long QT syndrome. In an embodiment, the effective amount is an amount to stabilize a cardiac membrane independent of QT change.

In embodiments where the cardioprotective agent is magnesium, potassium or calcium, the composition can include a salt of magnesium, potassium or calcium in an amount that provides a dose of the mineral (i.e., an elemental dose or amount) effective to achieve a physiologic effect on the QT interval, to shorten the QT interval, to reduce risk of developing a prolonged QT interval, to stabilize a cardiac membrane, and/or to reduce risks of a long QT interval. In an embodiment, the effective amount is an elemental amount to reduce risk of a drug-induced long QT syndrome.

In studies performed herein, and discussed below with reference to Example 1, the cardioprotective agent magnesium was administered to the subjects. The amount (weight) of magnesium salt in the composition will vary according to its salt form, as can be appreciated. In an embodiment, the composition comprises magnesium salt in an amount between about 50-8000 mg, 250-8000 mg, 500-8000 mg, 1000-8000 mg, 50-6000 mg, 250-6000 mg, 500-6000 mg, 1000-6000 mg, 1500-6000 mg, 50-5000 mg, 250-5000 mg, 500-5000 mg, 2000-5000 mg, 50-4000 mg, 250-4000 mg, or 500-4000 mg.

In other embodiments, the composition comprises an amount of a mineral salt, such as a potassium, a chloride, or a magnesium salt, that provides a dose of elemental mineral of between about 0.01-500 moles, 0.01-300 moles, 0.01-250 moles, 0.01-200 moles, 0.01-150 moles, 0.01-100 moles, 0.01-50 moles, 0.5-500 moles, 0.5-300 moles, 0.5-250 moles, 0.5-200 moles, 0.5-150 moles, 0.5-100 moles, 0.5-50 moles, 1-500 moles, 1-300 moles, 1-250 moles, 1-200 moles, 1-150 moles, 1-100 moles, 2-500 moles, 2-300 moles, 2-250 moles, 2-200 moles, 2-150 moles, 2-100 moles, 5-500 moles, 5-300 moles, 5-250 moles, 5-200 moles, 5-150 moles, 5-100 moles, 10-500 moles, 10-300 moles, 10-250 moles, 10-200 moles, 10-150 moles, or 10-100 moles. In other embodiments, the dose of elemental mineral provides between about 1-500 mEq, 1-300 mEq, 1-250 mEq, 1-200 mEq, 2-500 mEq, 2-300 mEq, 2-250 mEq, 2-200 mEq, 5-500 mEq, 5-300 mEq, 5-250 mEq, 5-200 mEq, or 5-150 mEq.

In an embodiment, the cardioprotective agent is magnesium administered in the form of a salt, such as magnesium sulfate or magnesium oxide.

The cardioprotective agent can be administered prior to, concurrently with, and/or subsequent to administration of the iboga alkaloid. The cardioprotective agent can be administered via any route of administration, and examples are discussed infra.

The composition comprises an amount of the iboga alkaloid or salt thereof in a amount to provide a desired therapeutic effect, as discussed herein with regard to treating neuropsychiatric disorders. Studies were performed, as discussed with reference to Example, with the iboga alkaloid ibogaine. More particularly, ibogaine in its hydrochloride salt form was used as a model agent to demonstrate the compositions and methods described herein. In an embodiment, the composition comprises a salt of ibogaine in an amount of between about 200-5000 mg, 200-3000 mg, 200-2500 mg, 200-2000 mg, 200-1800 mg, 200-1500 mg, 250-2500 mg, 250-2000 mg, 250-1800 mg, 250-1500 mg, 300-5000 mg, 300-3000 mg, 300-2500 mg, 300-2000 mg, 300-1800 mg, 300-1500 mg, 350-5000 mg, 350-3000 mg, 350-2500 mg, 350-2000 mg, 350-1800 mg, 350-1500 mg, 400-5000 mg, 400-3000 mg, 400-2500 mg, 400-2000 mg, 400-1800 mg, 400-1500 mg, 450-5000 mg, 450-3000 mg, 450-2500 mg, 450-2000 mg, 450-1800 mg, 450-1500 mg, 500-5000 mg, 500-3000 mg, 500-2500 mg, 500-2000 mg, 500-1800 mg, 500-1500 mg, 500-1600 mg, 500-1250, 550-5000 mg, 550-3000 mg, 550-2500 mg, 550-2000 mg, 550-1800 mg, 550-1600 mg, 550-1500 mg, 500-1250 mg, 600-5000 mg, 600-3000 mg, 600-1500 mg, 650-5000 mg, 650-3000 mg, 650-1500 mg, 650-1400 mg, 650-1400 mg, 700-5000 mg, 700-3000 mg, 700-2000 mg, 700-2500 mg, 700-1500 mg, 700-1400 mg, 700-1600 mg, 750-1600 mg, 750-1850 mg, 800-5000 mg, 800-3000 mg, 800-2500 mg, 800-2400 mg, 800-2250 mg, 800-2000 mg, 800-1800 mg, 800-1500 mg, 800-1600 mg, 800-1250, 850-5000 mg, 850-3000 mg, 850-2500 mg, 850-2400 mg, 850-2250 mg, 850-2000 mg, 850-1800 mg, 850-1600 mg, 850-1500 mg, or 850-1250 mg.

The composition comprising the iboga alkaloid and cardioprotective agent can be of any form. In embodiments, and as can be appreciated, the form of the composition may depend on the intended route of administration. In embodiments, the iboga alkaloid and cardioprotective agent are formulated together in a single dosage form. In embodiments, the iboga alkaloid and cardioprotective agent are formulated each in a single dosage form for administration sequentially or concurrently.

The composition can be a solid dosage form. In an embodiment, the solid dosage form in intended for oral ingestion. Examples of orally ingestible dosage forms include a powder, a capsule, a pill, or a tablet. In other embodiments, the solid dosage form is for sublingual, buccal, rectal or topical administration. The dosage form can be formulated to provide immediate release of one or both of the iboga alkaloid and cardioprotective agent upon oral administration. For example, a dosage form that releases the iboga alkaloid or salt thereof at least about 0.25 hours, 0.5 hours, 1 hour, 1.5 hours, 2 hours, 2.5 hours or 3 hours after release of the cardioprotective agent is contemplated.

An exemplary dosage form is an orally ingestible powder, capsule, pill, or tablet comprising an amount of an iboga alkaloid and an amount of a cardioprotective agent. In an embodiment, the iboga alkaloid is ibogaine. In an embodiment, the iboga alkaloid is a salt form of ibogaine, such as ibogaine hydrochloride. In an embodiment, the cardioprotective agent is an electrolyte, such as magnesium or any of the other minerals mentioned herein. The dosage form comprises an amount of ibogaine and an amount of cardioprotective agent in any of the amounts mentioned herein.

In embodiments, the composition can be suitable for a variety of delivery modes including, without limitation, oral, sublingual, buccal, intrapulmonary, or intranasal delivery. Compositions suitable for internal, rectal, vaginal, lingual, intravenous, intraarterial, intramuscular, intraperitoneal, intracutaneous and subcutaneous routes may also be used. Other dosage forms include tablets, capsules, pills, powders, aerosols, suppositories, parenterals, and oral liquids, including suspensions, solutions and emulsions. Sustained release dosage forms may also be used. All dosage forms may be prepared using methods that are standard in the art (see e.g., Remington's Pharmaceutical Sciences, 16th ed., A Oslo editor, Easton Pa. 1980).

In one embodiment, an iboga alkaloid or pharmaceutically acceptable salt or solvate thereof is administered orally, which may conveniently be provided in tablet, caplet, sublingual, liquid or capsule form. Solutions can be prepared using water or physiologically compatible organic solvents such as fatty alcohols, triglycerides, glycerine and the like. Parenteral compositions containing iboga alkaloid or pharmaceutically acceptable salt or solvate thereof may be prepared using conventional techniques that may include sterile isotonic saline, water, Ringer's solution, etc. Compositions for sublingual administration, for example as sublingual tablets may be designed to dissolve rapidly and can contain in addition to the iboga alkaloid and/or the cardioprotective agent, an excipients such as lactose, sucrose, dextrose or mannitol.

In other embodiments, a kit comprising a device comprising a plurality of unit doses of a cardioprotective agent and a unit dose of an iboga alkaloid or salt thereof and instructions for use is provided. In one embodiment, the plurality of unit doses of cardioprotective agent comprises a plurality of unit doses for oral administration of the cardioprotective agent. For example, each unit dose of cardioprotective agent in the kit can be a solid dosage form, such as a pill, capsule, tablet, caplet, or a powder. Each unit dose of cardioprotective agent, in other embodiments, can be a liquid dosage form, such as a solution, suspension, elixir, or syrup. In other embodiments, the plurality of unit doses of cardioprotective agent comprise a first set that is a solid dosage form and a second set that is a liquid dosage form; that is, the unit doses in the plurality are a mixture of solid dosage forms and liquid dosage forms. In embodiments, one or more of the unit doses is a solid, such as a powder, that is mixed with a liquid, such as water or juice, for ingestion by the patient. In other embodiments, one or more of the unit doses of cardioprotective agent is a liquid suitable for nasal administration or parenteral administration.

In embodiments, the kit or device of the kit comprises a unit dose of iboga alkaloid or salt thereof in the form of a solid or a liquid. The solid unit dose can be, for example, a pill, capsule, tablet, caplet, or a powder. The liquid unit dose can be, for example, an elixir, a syrup, a suspension or a solution. In other embodiments, the unit dose of iboga alkaloid or salt thereof is a liquid suitable for nasal administration or parenteral administration.

In an embodiment, the device comprising the unit dose of iboga alkaloid or salt thereof and/or the plurality of unit doses of cardioprotective agent is a blister pack. In other embodiments, the kit comprises a device comprising the unit dose of iboga alkaloid and a container or collection of packets with a plurality of doses of the cardioprotective agent. In other embodiments, the kit comprises a blister pack with at least one unit dose of iboga alkaloid or salt thereof and at least one unit dose of a cardioprotective agent. In other embodiments, the kit comprises a blister pack with at least one unit dose of iboga alkaloid or salt thereof and two or more unit dose of a cardioprotective agent. In embodiments, each unit dose in the blister pack is in a separate blister of the blister pack. By way of example, a kit comprising a plurality of doses of a magnesium salt, calcium salt or potassium salt (or combination of mineral salts) is provided, where in embodiments the plurality of doses are in a blister pack of individual unit doses, in a collection of containers each with a unit dose, or in a single container comprising the plurality of doses. The kit further comprises at least one dose of, for example, ibogaine hydrochloride.

In an embodiment, the device further comprises a second unit dose of the iboga alkaloid or salt thereof. In an embodiment, the second unit dose of the iboga alkaloid or salt thereof is at a dose different than the first unit dose or at the same dose as the first unit dose. In an embodiment, the second unit dose of the iboga alkaloid or salt thereof is at a dose less than the dose of the iboga alkaloid or salt thereof in the first unit dose. In an embodiment, the plurality of unit doses of cardioprotective agent comprise a plurality of unit doses at different doses and/or of unit doses formulated for different routes of administration.

### III. Methods of Treatment

In an aspect, a method for treating a neuropsychiatric disorder is provided. The method comprises administering, or instructing to administer, to a subject a cardioprotective agent in an amount effective to achieve a physiologic effect to reduce risk of long QT syndrome and administering an iboga alkaloid or salt thereof. In embodiments, the neuropsychiatric disorder is a neurological disorder or a neurodegenerative disease.

In an embodiment, the neuropsychiatric disorder is traumatic brain injury (TBI) and a method for treating TBI is provided. TBI is a leading cause of disability worldwide and is a signature injury of US Veterans from recent military conflicts, most often caused by blast exposure. Clinically, sequelae of TBI can include posttraumatic stress disorder (PTSD), major depressive Disorder (MDD), and anxiety disorders, but efficacy of treatments for these complications is limited. For example, first-line therapies for PTSD are less effective in veteran populations (Beidel, D. C. et al. Contemp. Clin. Trials Commun. 17, 100491 (2020); Bryan, C. J. et al., Psychol. Trauma Theory Res. Pract. Policy 10, 36-45 (2018); Steenkamp, M. M. et al., JAMA 323, 656-657 (2020)), and overall remission rates of available treatments for these complications range from 20-40% (Steenkamp, M. M. et al., JAMA 314, 489-500 (2015); Alexander, W., Pharm. Ther. 37, 32-38 (2012)). Perhaps most concerningly, veterans make up 20% of suicides in the US despite making up only 6.4% of the general population (Inoue, C. et al., StatPearls (StatPearls Publishing, 2022). As mentioned above, ibogaine is linked to neuro- and cardiotoxicity concerns (Litjens, R. P. W. et al., Clin. Toxicol. 54, 297-302 (2016); Ona, G. et al. Psychopharmacology (Berl.) 239, 1977-1987 (2022)). In regards to the former, only transient ataxia has been reported in humans (Litjens et al., supra). In the case of the latter, however, lengthening of the time of ventricular depolarization and repolarization (QT interval prolongation) with instances of subsequent fatal arrhythmia has occurred (Ona, G. et al., supra). High doses of ibogaine, pre-existing conditions, drug-drug interactions, and lack of vital sign monitoring may have played critical roles in these cases (Litjens et al., supra).

A study was conducted to evaluate safety and efficacy of ibogaine, as an exemplary iboga alkaloid, in special operations veterans with combat and/or blast exposure, as described in Example 1. Veterans are more likely to have chronic conditions. Substance use in four times higher in veterans than the general population, with an incidence of lifetime trauma a primary predictor of developing substance use disorder (Barsuglia et al., Prog. Brain. Res., 424:121-158 (2018)). Traumatic brain injury is a signature injury of veterans, with almost half (46%) of returning U.S. military personnel from conflicts in Iraq and Afghanistan screening positive for TBI (Morissette et al., Rehabil Psychol., 56(4):240-350 (2011)). Common mechanisms of injury include blasts, car accidents and falls (Schneiderman et al., Am J. Epidemiol, 167:144601452 (2008)). A majority of traumatic brain injuries are mild or moderate, and form the basis of most TBI research. Most patients with a mild or moderate TBI (mTBI) recover within weeks, but many develop symptoms that last for months or years (Vanderploeg et al, J. Clinical and Experimental Neuropsychology, 29(6):585-598 (2007)). In a sample of patients with mTBI three months after initial injury, 79% complained of at least one persistent symptom and 34% still had a functional disability (Rimel et al., Neurosurgery, 9(3):221-229 (1981)). TBI is highly comorbid with PTSD, with 33-39% of soldiers with mTBI having PTSD (Polusny et al., Arch Gen Psychiatry, 68(1):79-89 (2011)). Other estimates are that PTSD prevalence after TBI in the general population range from 1-50%. PTSD is the most common psychiatric condition among veterans with TBI. Psychosocial and functional impairments are common in both disorders, as psychological trauma often co-occurs with biomechanical trauma. Damage to the frontal cortex during TBI may make patients more vulnerable to uncontrolled anxiety and fear reactions. Patients suffering from TBI and PTSD tend to complain of more severe neurocognitive symptoms.

The symptoms of PTSD and TBI overlap in that both can present with one or more of irritability, cognitive deficits, insomnia, depression, fatigue and/or anxiety. Subjects with only TBI can present with one or more of headache, sensitivity to light or noise, nausea, vomiting, vision problems, and/or dizziness. Subjects with only PTSD can present with one or more of flashbacks, avoidance, hypervigilance, nightmares and/or re-experiencing phenomenon. Both TBI and PTSD are associated with suicidal ideation (Wisco et al., J. Clinical Psychiatry, 75(12):1338-1346 (2014)), and suicidal ideation is higher among veterans than civilians with almost one quarter of veterans having suicidal ideation. Veterans have the highest rate of death by suicide compared to military and general populations, and of suicides between 2012-2015 in veterans, nearly all reported distress stemming from a form of emotional trauma following a first deployment.

As described in Example 1, subjects that were special operations veterans were enrolled for treatment with ibogaine and psychological testing, neuropsychological testing, fMRI, EEG and self-report measures. The pre-specified primary outcome was change in the World Health Organization Disability Assessment Schedule 2.0 (WHODAS) (Gold L. H. et al., J. Am. Acad. Psychiatry Law 42, 9 (2014)) from baseline to post-treatment, with change from baseline to the one-month follow-up a secondary outcome. Additional pre-specified secondary outcomes included post-treatment changes on the Clinician Administered PTSD Scale (CAPS-5; Weathers, F. W. et al., Psychol. Assess., 30, 383-395 (2018)), Montgomery-Asberg Depression Rating Scale (MADRS; Montgomery, S. A., Br. J. Psychiatry J. Ment. Sci., 134, 382-389 (1979)) and the Hamilton-Anxiety Rating Scale (HAM-A; Bruss, G. S., et al., Psychiatry Res., 53, 191-202 (1994)). All participants were screened medically by the treatment site, and medications with potentially concerning drug-drug interactions were discontinued prior to treatment. On the day of treatment, participants were administered one gram of prophylactic magnesium sulfate intravenously and 12.1±1.2 (mean±SD) mg/kg of oral ibogaine while under medical supervision. An additional dose of magnesium was administered approximately 12 hours later. To assess the significance of post-treatment changes, linear mixed effects (LME) models were used for each outcome measure. False Discovery Rate (FDR; Benjamini, Y. et al., J. R. Stat. Soc. Ser. B Methodol., 57, 289-300 (1995)) was applied to correct for multiple comparisons.

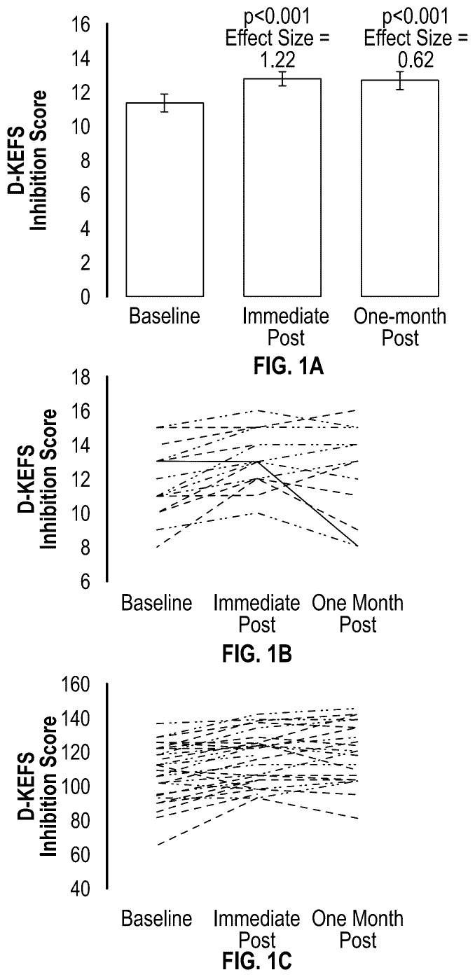

The results are shown in Table 1, FIGS. 1-9 and Tables 1-2, 1-3, 1-4, 1-5 and 1-6 in Example 1. For simplicity, reference herein to “post treatment”, “after treatment”, “post ibogaine treatment” and “after ibogaine treatment”, and variations of these phrases, refer to the period subsequent to the treatment regimen of Example 1 where a cardioprotective agent was administered in conjunction with (e.g., prior to, during or after administration of) an iboga alkaloid, exemplified by ibogaine. Reference to ‘immediate post’ treatment, ‘4 days post treatment’ and ‘1 week post treatment’ refer to the first assessment made post treatment with ibogaine (as an exemplary iboga alkaloid) and a cardioprotective agent, which was typically 4 days after ibogaine administration or in some cases 1 week after ibogaine administration. Reference to “one-month post treatment” refers to the assessment made one month after treatment with ibogaine (as an exemplary iboga alkaloid) and cardioprotective agent.

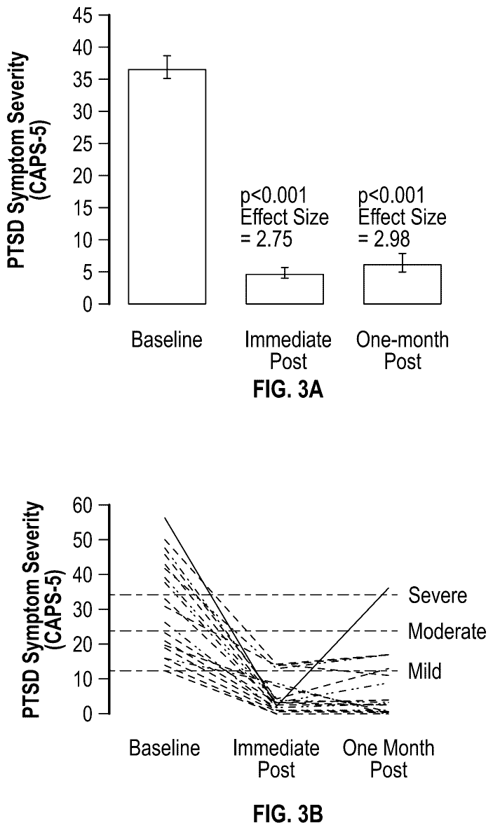

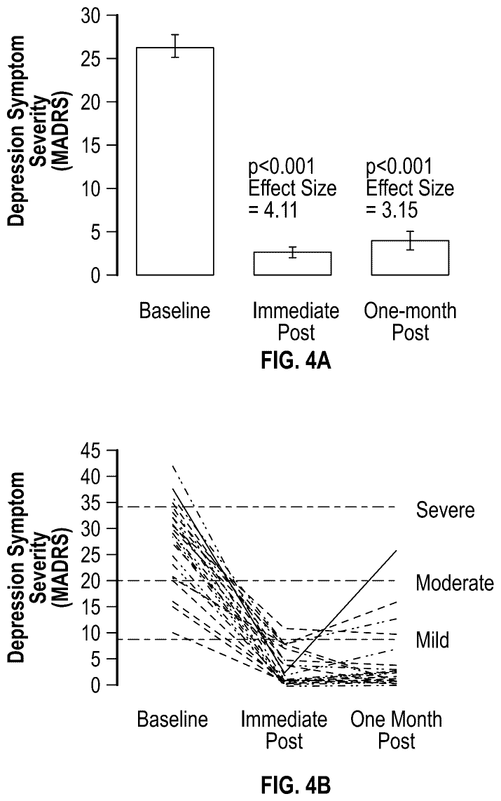

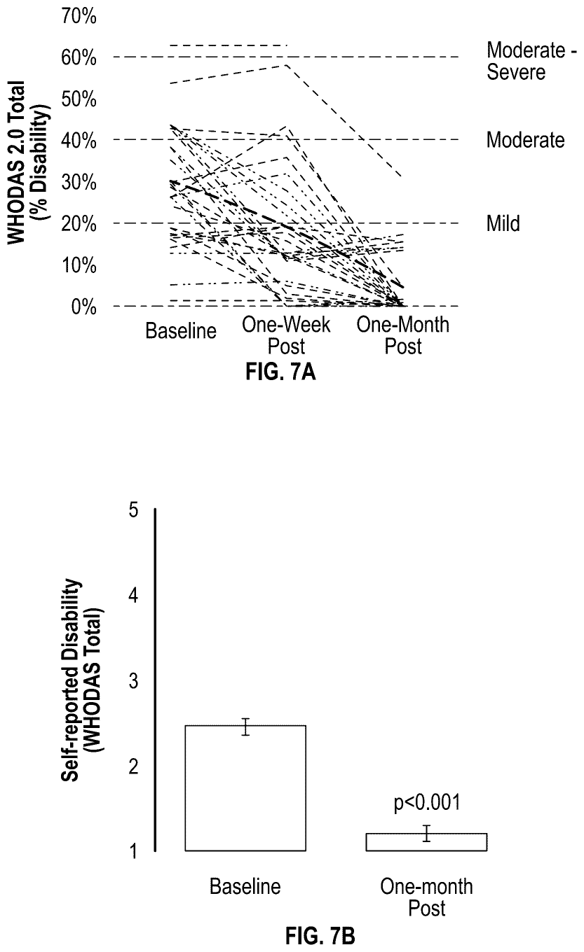

As seen in Table 1, there was a significant decrease of WHODAS total score from baseline to the immediate post evaluation and to the one-month follow-up (FIG. 7A) with effect sizes (Cohen's D) of 0.74 and 2.20, respectively. The improvement was statistically significant across all subscales (Table 1-1 in Example 1), with the greatest effect size noted for the cognition domain (D=2.38). The models also revealed significantly lowered CAPS-5, MADRS, and HAM-A scores one-week post treatment and at the one-month follow-up (FIG. 3B, FIG. 4B, Table 1).

To further assess changes in comorbid psychiatric symptoms identified by the models, mean percent reduction, response rate, and remission rate according to the CAPS-5, MADRS and HAM-A (Table 1) were calculated. Response on the CAPS-5, MADRS, and HAM-A was defined as a reduction of at least 10 points (Mitchell, J. M. et al., Nat. Med. 27, 1025-1033 (2021)), 50% (Leucht, S. et al., J. Affect. Disord. 210, 287-293 (2017)), and 50% (McIntyre, A. et al., Depress. Anxiety 24, 487-494 (2007)), respectively; remission as loss of diagnosis and a total score below 12 (Mitchell, J. M. et al. supra), <8 (Leucht, S. et al. supra), and <8 (McIntyre, A. et al., supra), respectively. One participant's baseline scores met criteria for remission in all three scales and so were excluded from the calculation of response and remission rates, leaving 29 participants in these specific analyses. As shown in FIG. 3B, FIG. 4B, and FIG. 7A and in Table 1, mean percent reductions were at least 81%, response rates were at least 93%, and remission rates were at least 83%; effect sizes were all above 2.0.

TABLE 1
Demographics and Results of Study in Example 1

Baseline Demographics

| | |
|---|---|
| & Characteristics | Diagnosis According to MINI DSM-5 |

| | | | |
|---|---|---|---|
| Total N | 30 | | N |
| Age | 44.9 ± 7.5 | Post-Traumatic Stress Disorder | 23 |
| TBI (Mild, | 28, 1, 1 | Major Depressive Disorder | 15 |
| Moderate, | | | |
| Moderately | | | |
| Severe)^{a} | | | |
| Combat | 29.6 ± 5.2 | Anxiety Disorders^{d} | 15 |
| Exposure | | | |
| Scale^{b} | | | |
| Number of | 38.6 ± 52.4 | Alcohol Use Disorder | 15 |
| TBIs^{c} | | | |
| Number | 5.5 ± 3.0 | Other Substance Use Disorders^{f} | 6 |
| Combat | | | |
| Deployments | | | |
| Time Since | 7.7 ± 4.8 | Race and Ethnicity | |
| Military | | | |
| Discharge | | | |
| Past Suicidal | 19 | White | 26 |
| Ideation | | Biracial (White and Native American) | 2 |
| Past Suicide | 7 | Native American | 1 |
| Attempt | | Hispanic | 1 |

Baseline and Follow-up Statistics

| | | One- | Baseline vs One- | | | One- | Baseline vs One- | | |
|---|---|---|---|---|---|---|---|---|---|
| | | Week | week post | | | Month | Month post | | |
| | Baseline | Post | F^{e} | p(FDR) | D | Post | F^{e} | p(FDR) | D |
| WHODAS | 30.2 ± | 19.9 ± | 20.34 | <0.001 | 0.74 | 5.1 ± | 85.86 | <0.001 | 2.20 |
| 2.0 Total | 14.7 | 16.3 | | | | 8.1 | | | |
| CAPS-5 | 31.7 ± | 3.9 ± | 206.14 | <0.001 | 2.30 | 4.8 ± | 191.77 | <0.001 | 2.54 |
| | 12.5 | 4.8 | | | | 7.9 | | | |
| MADRS | 25.6 ± | 2.8 ± | 249.72 | <0.001 | 2.65 | 3.8 ± | 229.28 | <0.001 | 2.80 |
| | 8.7 | 3.3 | | | | 6.0 | | | |
| HAM-A | 20.8 ± | 3.6 ± | 164.24 | <0.001 | 2.06 | 3.9 ± | 164.24 | <0.001 | 2.13 |
| | 8.5 | 3.4 | | | | 4.6 | | | |
| | % | % | X² | p(FDR) | — | % | X² | p(FDR) | — |
| | Reporting | Reporting | | | | Reporting | | | |
| | SI | SI | | | | SI | | | |
| Suicidal | 47% | 0% | 18.26 | <0.001 | — | 7% | 12.27 | 0.001 | — |
| Ideation | | | | | | | | | |
| (MADRS | | | | | | | | | |
| Q10) | | | | | | | | | |

Percent Reduction, Response and Remission Rates

| % Reduction vs | | |
|---|---|---|
| Baseline | Response Rate | Remission Rate |

| | One- | One- | One- | One- | One- | One- |
|---|---|---|---|---|---|---|
| | Week | Month | Week | Month | Week | Month |
| | Post | Post | Post | Post | Post | Post |
| CAPS-5 | 88% ± | 88% ± | 97% | 100% | 86% | 86% |
| | 15% | 17% | | | | |
| MADRS | 87% ± | 87% ± | 100% | 97% | 83% | 83% |
| | 23% | 17% | | | | |
| HAM-A | 81% ± | 81% ± | 97% | 93% | 86% | 83% |
| | 19% | 21% | | | | |

*All results presented as mean ± SD. SD = Standard Deviation; MINI = Mini International Neuropsychiatric Interview; DSM-5 = Diagnostic and Statistical Manual of Mental Disorders, Fifth Edition;
^{a}TBI severity was assessed using the Ohio State University TBI Identification Method;
^{b}A higher score on Combat Exposure Scale means higher combat-related stress;
^{c}Number of TBIs was assessed using Boston Assessment of Traumatic Brain Injury- Lifetime (BAT-L);
^{d}Includes panic disorder (8), panic attacks (4), social anxiety disorder (2), and agoraphobia (1);
^{e}Degrees of freedom (df) were (1, 75) for WHODAS-2.0 and (1, 78) for CAPS-5, MADRS, and HAM-A;
^{f}Includes pain medication (1), stimulants (3), and THC (2).

To ensure that individuals without the relevant comorbidity were not driving the findings, sensitivity analyses were performed, repeating these calculations in subgroups that excluded all participants who did not meet criteria on structured diagnostic interview for the disorder assessed by the scale (i.e., PTSD for CAPS-5). The results were similar, with remission rates at one-month follow up of at least 67% (Table 1-3 in Example 1). To determine if the participants with more severe TBI history may be biasing results, a sensitivity analysis excluding the participants with non-mild TBI; results was done, again with the results largely unchanged (Table 1-4 in Example 1), with remission rates at one-month follow up of at least 85%.

An analysis of the effect of ibogaine treatment on suicidal ideation was also performed. The proportion of participants with a score of one or above on the MADRS suicidal ideation item were compared and a statistically significant reduction from 47% at baseline to 0% and 7% at post-treatment and one-month follow-up, respectively, was found (Table 1). Results were similar when including only participants with measurable suicidal ideation at baseline (endorsing >1), and when excluding participants with non-mild TBI (Tables 1-3 and 1-4 in Example 1).

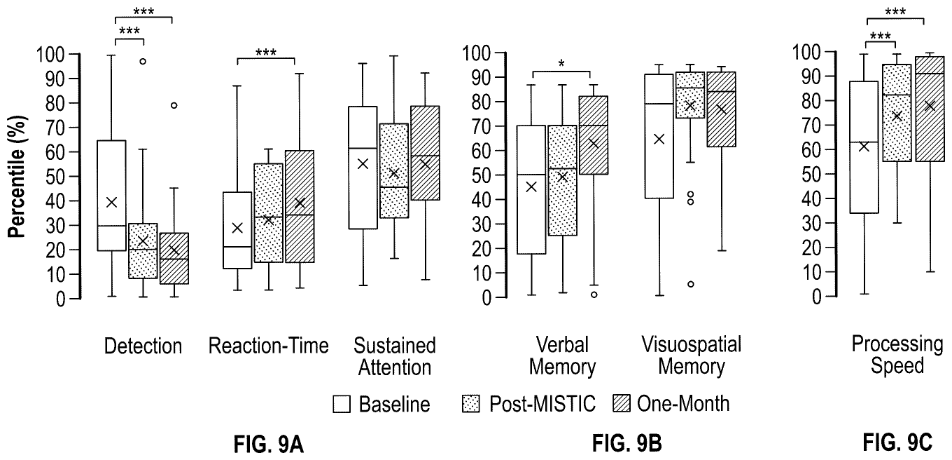

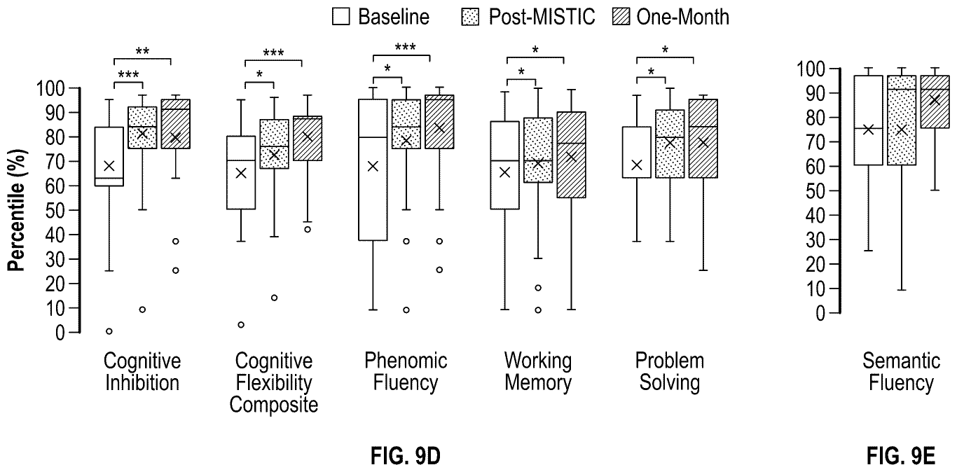

To assess for cognitive effects of ibogaine in combination with a cardioprotective agent, particularly given the history of TBI in study participants, a neuropsychological battery was administered to participants at all three timepoints. A summary of the results is in Table 2, Table 1-6 of Example 1 and FIGS. 9A-9E, showing pre-post score comparisons. Results indicated statistically significant improvements in processing speed with large effect sizes (D=0.97-1.34) and executive functioning (including inhibition, cognitive flexibility, problem-solving, phonemic fluency, and working memory; with effects ranging from small to large: D=0.31-1.22) both immediately post-ibogaine and at the one-month follow-up. Mean performances on these tests moved from the Average to High Average score range relative to age peers in all but one instance (phonemic fluency was High Average at baseline and improved to the Superior range relative to age peers at the one-month follow-up; (D=1.11). Learning and memory tests showed a significant improvement in visual memory at both timepoints and in verbal memory at the one-month follow-up. Sustained attention showed a significant improvement in accuracy (detection) at both time points with large effect sizes (D=0.86-1.05), and a weak but significant slowing of reaction time (D=0.29-0.52), consistent with a prioritization of accuracy over speed and reduced impulsivity. No significant performance changes were observed in language (semantic fluency). No declines were noted across any performance domain. In Table 2, neuropsychological test scores are represented as a T score (mean of 50, SD of 10). Unless stated otherwise, a higher score represents better performance.

TABLE 2
Baseline and follow-up statistics of neuropsychological testing. All results presented as mean ± SD.

| | | | | Baseline vs | | | | Baseline vs | | |
|---|---|---|---|---|---|---|---|---|---|---|
| | | | 1-wk | 1-wk post | | | 1-mo. | 1-month post | | |
| Neuropsych.Construct | Test Item | Baseline | post | F^{a} | p(FDR) | D | post | F^{a} | p(FDR) | D |

Sustained Attention

| | | | | | | | | | | |
|---|---|---|---|---|---|---|---|---|---|---|
| Detection^{b} | CPT-3 Detection | 46.6 ± 10.3 | 41.2 ± 8.2 | 13.11 | 0.002* | 1.05 | 39.5 ± 7.5 | 19.90 | <0.001* | 0.86 |
| Reaction Time | CPT-3 Reaction Time | 43.0 ± 7.7 | 44.1 ± 6.8 | 1.38 | 0.285 | 0.29 | 46.4 ± 8.1 | 9.49 | 0.007* | 0.52 |
| Sustained Attention | CPT-3 Hit Reaction | 51.5 ± 8.8 | 50.8 ± 7.9 | 0.01 | 0.952 | 0.02 | 51.2 ± 7.7 | 0.39 | 0.583 | 0.29 |
| | Time Block Change | | | | | | | | | |

Learning and Memory

| | | | | | | | | | | |
|---|---|---|---|---|---|---|---|---|---|---|
| Verbal Memory | HVLT-R | 47.4 ± 10.1 | 49.0 ± 9.2 | 0.42 | 0.583 | 0.17 | 53.1 ± 8.8 | 6.47 | 0.024* | 0.47 |
| Visuospatial Memory | BVMT-R | 53.9 ± 11.4 | 58.8 ± 7.1 | 9.73 | 0.007 | 0.50 | 58.3 ± 6.6 | 4.41 | 0.052 | 0.32 |

Processing speed

| | | | | | | | | | | |
|---|---|---|---|---|---|---|---|---|---|---|
| Processing Speed | PSI (WAIS-IV) | 53.8 ± 10.6 | 59.2 ± 9.7 | 27.85 | <0.001* | 0.97 | 61.6 ± 10.7 | 43.43 | <0.001* | 1.34 |

Executive Function

| | | | | | | | | | | |
|---|---|---|---|---|---|---|---|---|---|---|
| Cognitive Inhibition | D-KEFS Color/Word | 55.1 ± 8.8 | 59.9 ± 6.4 | 21.30 | <0.001* | 1.22 | 59.9 ± 7.5 | 15.79 | 0.001* | 0.62 |
| | Interference, | | | | | | | | | |
| | Condition 3 | | | | | | | | | |
| Cognitive Flexibility | Average of: (1) D- | 54.0 ± 8.0 | 56.6 ± 5.7 | 4.73 | 0.045* | 0.43 | 59.3 ± 5.0 | 17.62 | <0.001* | 0.74 |
| Composite | KEFS TMT, | | | | | | | | | |
| | Condition 4; (2) D- | | | | | | | | | |
| | KEFS Color/Word | | | | | | | | | |
| | Interference, | | | | | | | | | |
| | Condition 4; (3) D- | | | | | | | | | |
| | KEFS Verbal Fluency, | | | | | | | | | |
| | Category Switching | | | | | | | | | |
| Phonemic Fluency | D-KEFS Verbal | 57.0 ± 11.7 | 60.8 ± 10.3 | 7.53 | 0.016* | 0.52 | 64.0 ± 10.1 | 21.99 | <0.001* | 1.11 |
| | Fluency | | | | | | | | | |
| Working Memory | WMI (WAIS-IV) | 55.1 ± 8.3 | 57.0 ± 9.5 | 5.32 | 0.034* | 0.37 | 57.6 ± 9.2 | 6.02 | 0.027* | 0.31 |
| Problem Solving | D-KEFS TT, Total | 55.7 ± 6.4 | 59.1 ± 7.1 | 5.38 | 0.034* | 0.49 | 59.5 ± 7.9 | 6.42 | 0.024* | 0.44 |
| | Achievement Score | | | | | | | | | |

Language

| | | | | | | | | | | |
|---|---|---|---|---|---|---|---|---|---|---|
| Semantic Fluency | D-KEFS Verbal | 60.4 ± 11.4 | 60.2 ± 12.2 | 0.18 | 0.688 | 0.02 | 63.6 ± 7.8 | 2.00 | 0.200 | 0.24 |
| | Fluency | | | | | | | | | |

^{a}Degrees of freedom (df) were (1, 63) for CPT3, (1, 75) for working memory, verbal memory, and problem solving; and (1, 76) for visuospatial memory, cognitive inhibition, cognitive flexibility, phonemic fluency, semantic fluency, and processing speed.
^{b}Lower score indicates better performance.
SD = Standard Deviation; CPT-3 = Conners Continuous Performance Test; HVLT-R = Hopkins Verbal Learning Test-Revised; BVMT-R = Brief Visuospatial Memory Test-Revised; PSI = Processing Speed Index; WAIS-IV = Wechsler Adult Intelligence Scale 4th Edition; D-KEFS = Delis-Kaplan Executive Function System; TMT = Trail Making Test; WMI = Working Memory Index; TT = Tower Test.

With regard to safety, there were no unexpected or serious treatment-emergent side effects, and there were no instances of a concerning prolongation of QTc. All participants experienced transient cerebellar signs such as mild ataxia and intention tremor that resolved within 24 hours. While experiencing the oneirogenic effects of ibogaine, 12 participants (40%) were treated for headache, 7 (23%) for nausea, 3 (10%) for anxiety, 2 (7%) for hypertension, and 1 (3%) for insomnia.

The data from the study of Example 1 revealed clinically and statistically significant improvements in functioning both immediately and one-month post-treatment with an iboga alkaloid, exemplified by ibogaine, and a cardioprotective agent. Secondary analyses showed that remission rates for comorbid PTSD, depression, and anxiety were around 80%. There were no unexpected or serious adverse events. The safety and efficacy ofibogaine treatment in combination with a cardioprotective agent was evaluated in subjects with a history of TBI. At baseline, study participants were experiencing clinically significant levels of disability, PTSD, depression, and anxiety. Following ibogaine treatment in combination with a cardioprotective agent, participants showed a remarkable reduction in these symptoms with large effect sizes (Cohen's D>2 on clinician rated psychiatric assessments), and the benefits were sustained at the one-month follow-up. Indeed, disability measures continued to improve, and psychiatric symptom remission and response rates one-month post-treatment remained high. With regard to safety, no serious or unexpected adverse events occurred, and management of AEs was uncomplicated. The study demonstrates the potential of ibogaine in combination with a cardioprotective agent to be a powerful therapeutic for the transdiagnostic psychiatric symptoms that can emerge following TBI, including suicidality. Considering that the average time since discharge from the military in the tested subjects was nearly eight years, these findings further suggest that ibogaine in combination with a cardioprotective agent is effective even when administered years following the injuries.

Functional magnetic resonance imaging (fMRI) was performed on the subjects enrolled in the study described in Example 1. Neuroimaging is a branch of medical imaging that focuses on the brain. It utilizes techniques of physics, optics, and mathematics to quantify brain structure and function. Most neuroimaging techniques are non-invasive or minimally invasive, and are used to diagnose and assess brain health and function. fMRI provides an indirect measure of hemodynamic response blood oxygenation level dependency and is underpinned by the theory that if metabolic resources are being allocated to areas, it suggests that neurons in that area must be firing and this means activation. fMRI observes time dependent changes in blood flow. During resting, subjects are asked to do nothing and the aim is to observe the brain at rest. When the subject is asked to perform a task, the aim is to observe how the brain works in response to the task. Other magnetic resonance based imaging techniques include: anatomical, which measures cortical thickness, area, gyrification, and volume; arterial spin labelling, which quantitatively measures tissue perfusion, cerebral blood flow, where perfusion refers to the delivery of oxygen and nutrients to tissue by means of blood flow; and, diffusion tensor imaging, which measures diffusion of water through white natter, such as neural fiber tracts. fMRI can provide a series of images that form a film, as opposed to a single image obtained in MRI.

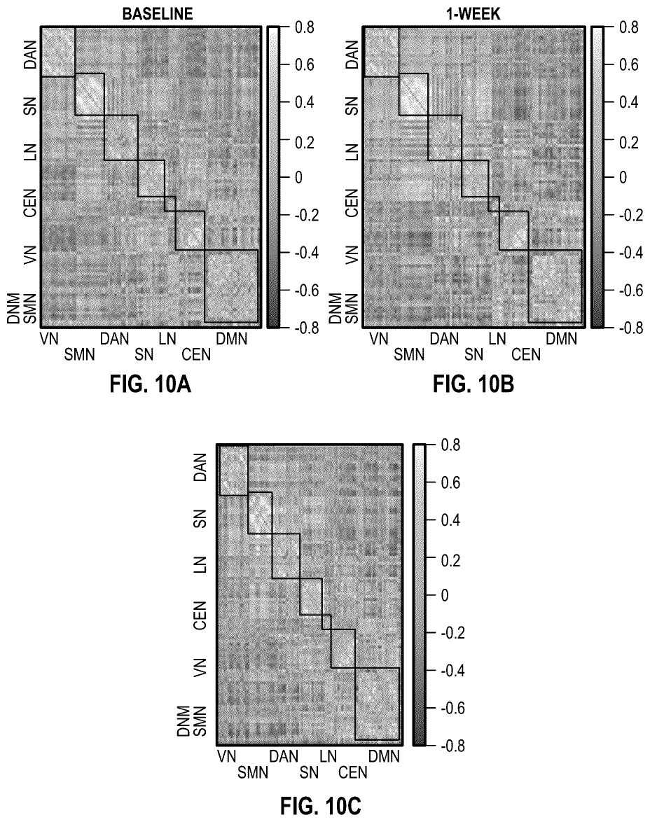

The human brain is a series of well-organized and optimized series of efficient networks. Disease states can be characterized as abnormalities or inefficiencies in the networks and/or the global organization. Resting-state functional connectivity is one method to characterize disease states and effects of interventions. The functional connectivity networks evaluated in a region of interest in the brain included those identified in Table 2-1 of Example 2. Data from the fMRI study is presented in FIGS. 10-16.

FIGS. 10A-10C show a correlation matrix of functional networks in the brain, as measured using functional MRI, in subjects at baseline, one week after treatment and one month after treatment.

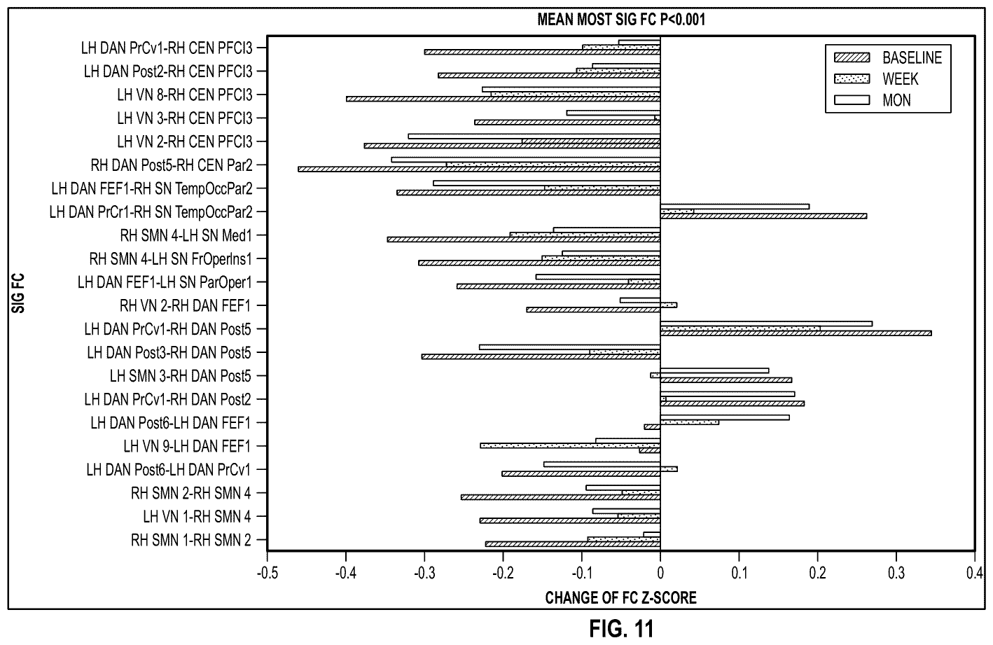

FIG. 11 is a graph showing significant changes in brain functional connectivity in Z-scores for subjects at baseline, one week after treatment and one month after treatment.

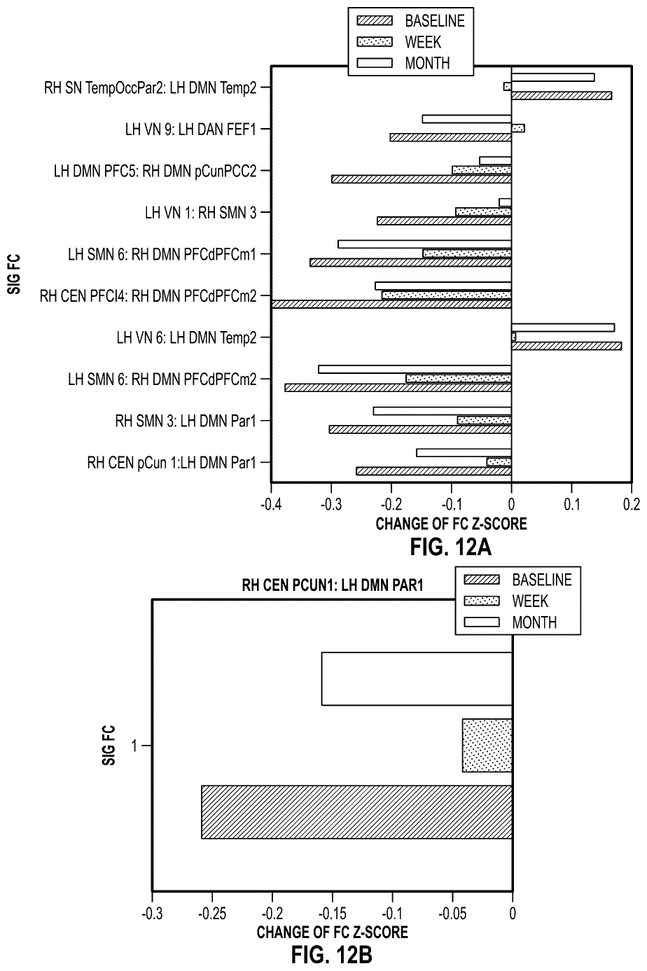

FIGS. 12A-12B are graphs showing significant changes in functional connectivity in Z-scores for subjects at baseline, one week after treatment and one month after treatment. FIG. 12B shows the change in functional connectivity of the right hemisphere (RH) central executive network (CEN) in the precuneus brain region to the left hemisphere (LH) default mode network (DMN) in the parietal brain region.

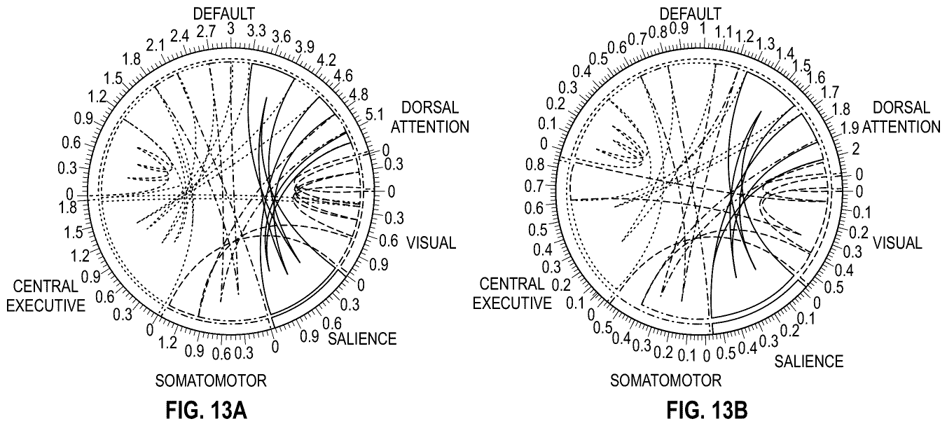

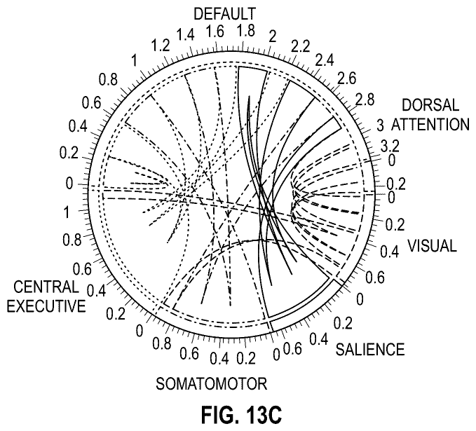

FIGS. 13A-13C provide visual representations of certain functional connectivity pairs in the brain network in subjects at baseline (FIG. 13A), one week after treatment (FIG. 13B), and one month after treatment (FIG. 13C), demonstrating changes in functional connectivity after treatment.

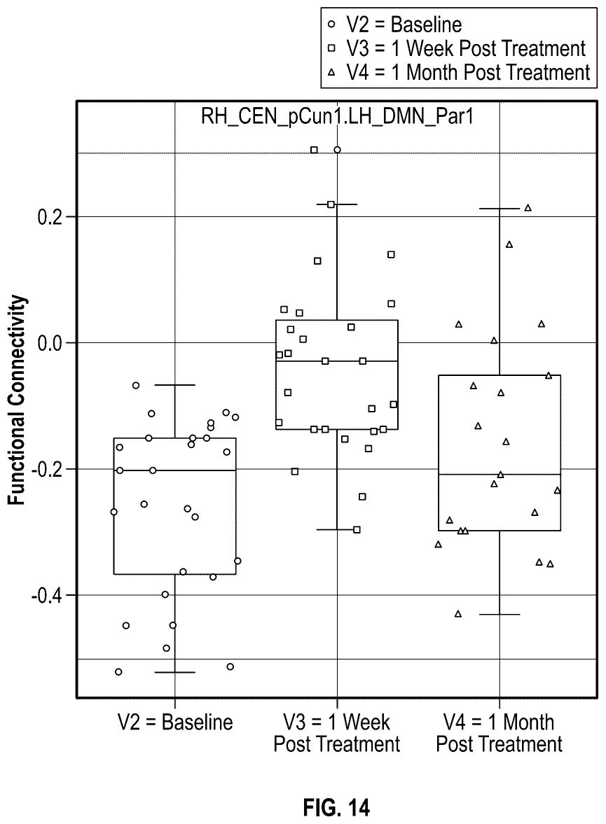

FIG. 14 is a graph showing group wide distribution of the functional connectivity of the right hemisphere (RH) central executive network (CEN) in the precuneus brain region to the left hemisphere (LH) default mode network (DMN) in the parietal brain region in subjects at baseline (v2), one week after treatment with ibogaine (v3) and one month after treatment (v4).

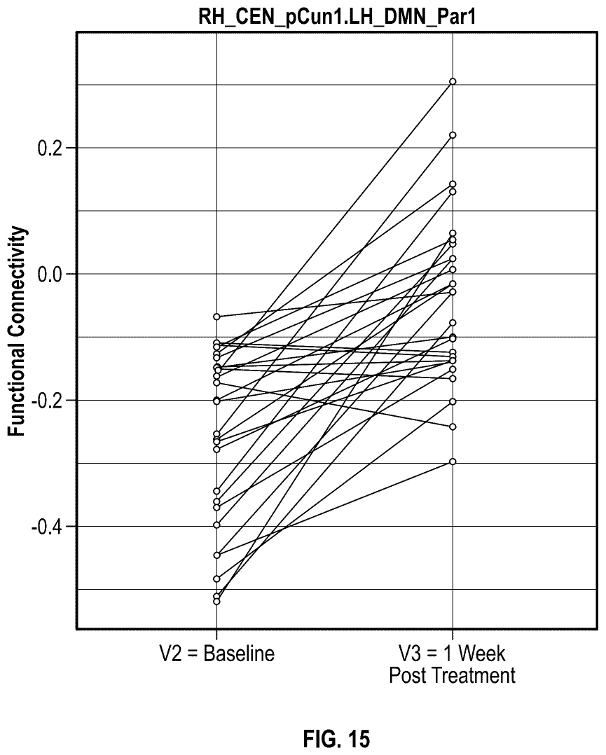

FIG. 15 is a graph showing changes in individual subjects of the functional connectivity of the right hemisphere (RH) central executive network (CEN) in the precuneus brain region to the left hemisphere (LH) default mode network (DMN) in the parietal brain region in subjects at baseline (v2) and one week after treatment (v3).

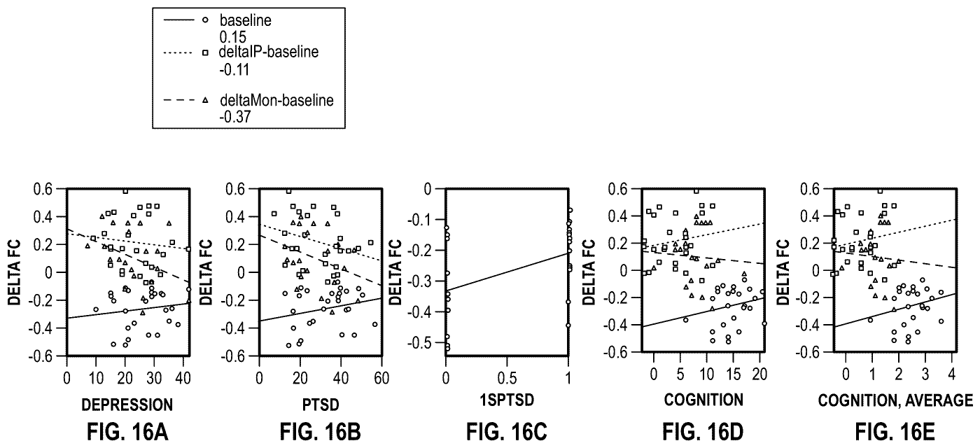

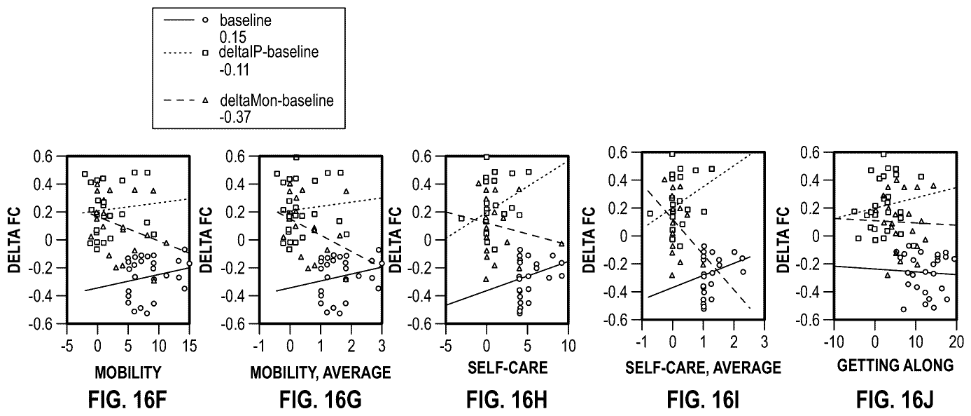

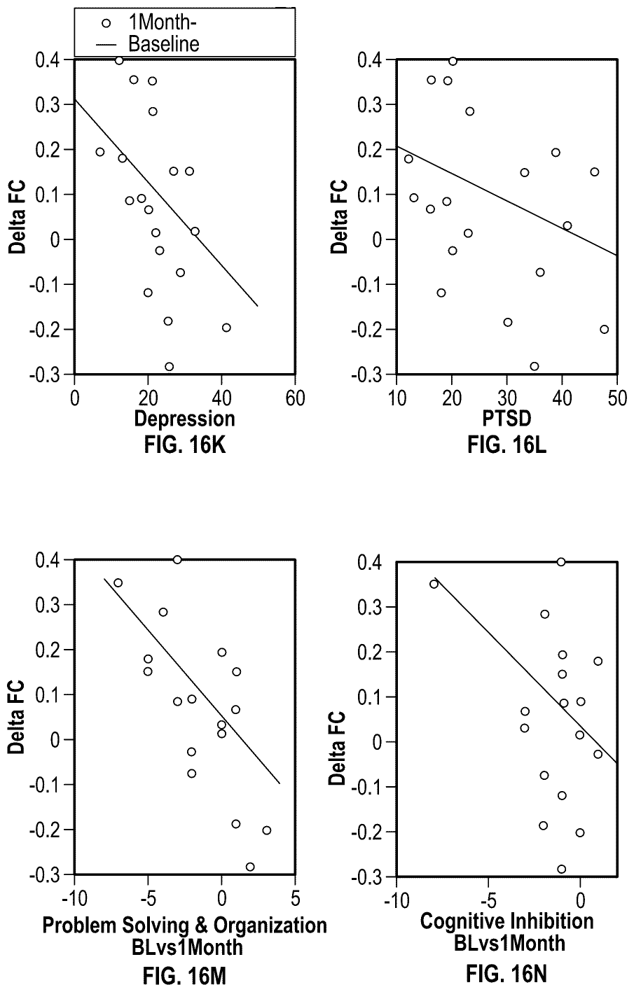

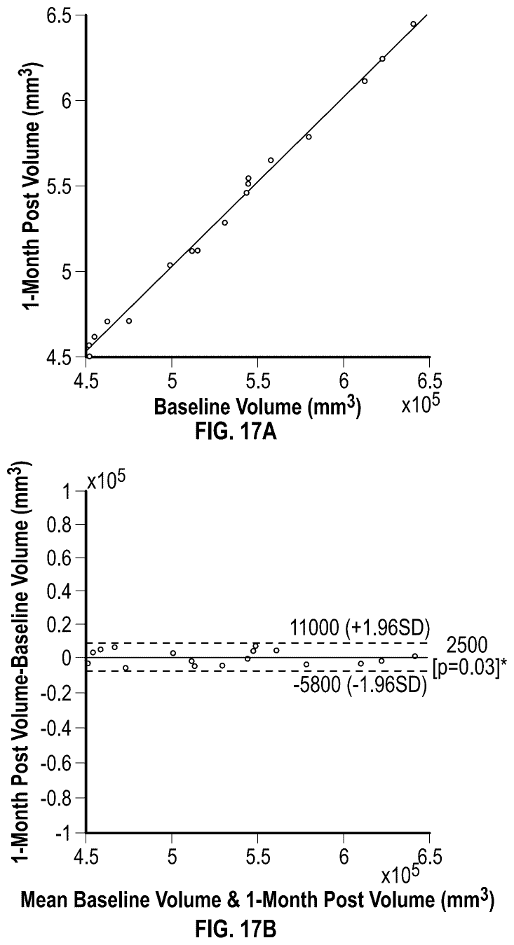

Resting state functional connectivity (rsFC) is a method to characterize how a disease state or a therapeutic intervention impacts brain networks. The right hemisphere central executive network (CEN) includes the precuneus region, where memory integration, episodic memory, mental imagery and pain occur. The left hemisphere default mode network (DMN) includes the parietal region, where somatosensory preprocessing occurs. Brain connectivity was correlated with clinical scores from the subjects in the study of Example 1, where change in functional connectivity of right hemisphere CEN to left hemisphere DMN at one month post treatment from baseline and one week post treatment from baseline were correlated with change in clinical scores at the same time periods. Results in are FIGS. 16A-16N.

FIGS. 16A-16J are graphs showing correlation between functional connectivity of the right hemisphere (RH) central executive network (CEN) in the precuneus brain region and the left hemisphere (LH) default mode network (DMN) in the parietal brain region and the clinical measure indicated on the x-axis of each graph. Values of the clinical measure for baseline are shown (open circles, solid line) and the change in functional connectivity (“delta FC”) from baseline and 1-week post treatment (squares, dotted line) and 1-month post treatment (triangles, dashed line) are shown for: depression symptom severity as measured using MADRS (FIG. 16A), PTSD symptoms assessed using CAPS-5 (FIG. 16B); in subjects with PTSD symptoms (1sPTSD) assessed using CAPS-5 (FIG. 16C), and WHODAS assessments of cognition (FIG. 16D), cognition average (FIG. 16E), mobility (FIG. 16F), mobility average (FIG. 16G), self-care (FIG. 16H), self-care average (FIG. 16I) and getting along (FIG. 16J).

FIGS. 16K-16N are bar graphs showing correlations of functional connectivity of right hemisphere CEN to left hemisphere DMN at one month post treatment from baseline and change in clinical scores for depression (FIG. 16K), PTSD (FIG. 16L), problem solving and organization (FIG. 16M) and cognitive inhibition (FIG. 16N). A decrease in functional connectivity between right hemisphere CEN and left hemisphere DMN predicts clinical improvement and improved performance in PTSD symptoms and problem solving task.

The data in FIGS. 1-16 demonstrate the resting-state functional connectivity (or differences in such connectivity pre- to post-treatment) observed between and within the most commonly described large scale brain networks as well as specific brain structures of interest. The data in Example 2 demonstrates that ibogaine as an intervention changes the functional connectivity architecture of the subjects.

fMRI data was also analyzed to inspect activity patterns as a function of time. A lag projection analysis measures temporal dynamics of blood oxygen level-dependent (BOLD) fMRI (Mitra et al., PNAS, E2235-E2244, 2015). Directed flow transfer of information is analyzed by comparing lag-correlation shift between voxel pairs. The average lag structure of a voxel indicates relative earliness or lateness in the brain, as a measure of whether the region is a source or sink of information. A paired T-test of fMRI images at baseline and about four days post treatment was done. A relative slowing in the following regions was observed (fwec p<0.001): left orbitofrontal gyrus (lateral and anterior) and inferior frontal gyrus (orbital and triangular, i.e., pars triangularis). These regions are involved in language formation, response inhibition and regulating goal-directed behavior. Slowing may reflect sustained processing and influence over the brain across long time scales.

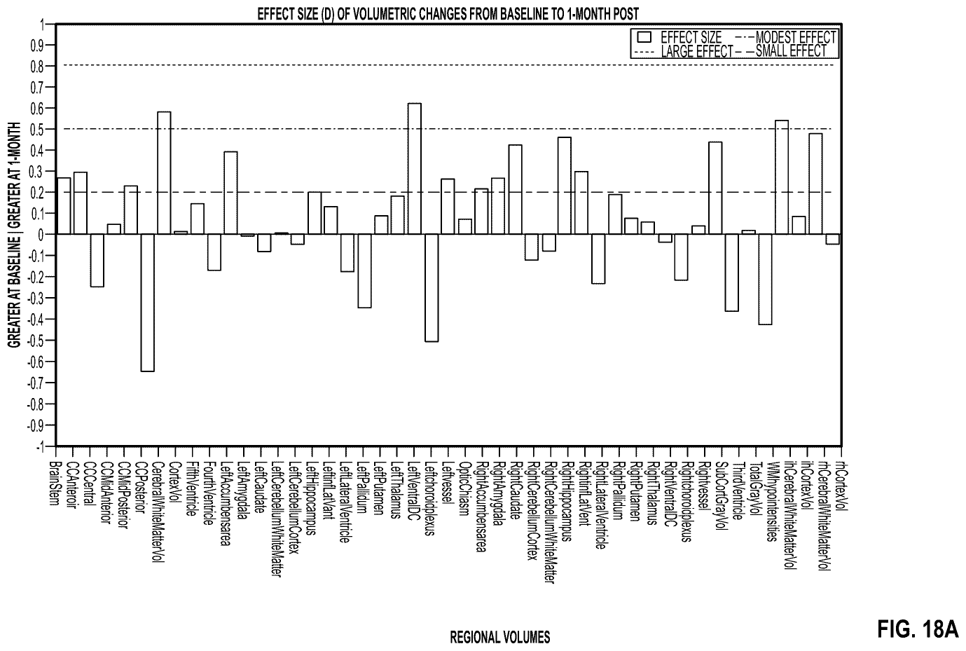

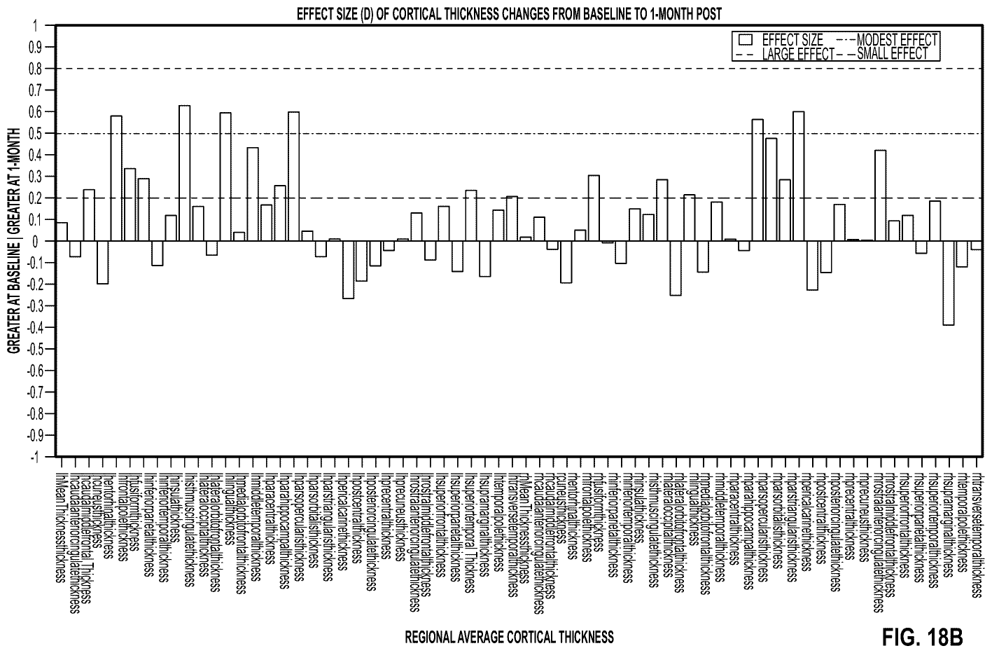

Structural imaging of the brain was also conducted on some of the subjects of Example 1. Gray matter constitutes about 40% of the brain and white matter constitutes about 60% of the brain. Gray matter contains most of the brain's neuronal cell nodes, it is fully developed once a person reaches their 20's and interprets sensor information from various parts of the body. White matter is made up of bundles which connect various gray matter areas, it develops throughout the 20's and peaks in middle age, Typical metrics analyzed in anatomical (T1-weighted, “T1w”) imaging include volume and/or shape of nuclei (e.g. caudate), volume and/or shape of ventricles of the brain, and cortical thickness (mm). These metrics can be used to study normal and abnormal neuroanatomy and are primarily used to quantify gray matter. A tool called FreeSurfer was applied to images for a T1w structural analysis (volume, cortical thickness) to adjust for bias, remove non-brain matter, segments by tissue, parcels the brain into standardized regions, and provides measurements of those regions. FreeSurfer measurements of whole brain volume, volume by tissue (gray, white, CSF), estimated intracranial volume (ICV), subcortical gray matter volume, cortical volume by region were done. Regional volume and cortical thickness were estimated at baseline and 1-month post treatment in 17 of the enrolled test subjects to probe for loss of brain tissue and/or indications of damaging effects after treatment with ibogaine. In particular, analysis was done to determine if there was any loss in gray matter volume or cortical thickness, whether there was a loss in white matter volume or an increase in presence of white matter hypotensities. Results are summarized in Table 3 and shown in FIGS. 17A-17B, and FIGS. 18A-18B.

TABLE 3
Whole Brain Index Volumes - Baseline
vs. one month post treatment

| Region of Interest | Statistical Analysis |
|---|---|
| Total Gray Matter | Two-tailed: no significant differences |
| | (p = 0.4755; alpha = 0.05) |
| Cortical Gray Matter | Two-tailed: no significant differences |
| | (p = 0.4847; alpha = 0.05) |
| Subcortical Gray Matter | Two-tailed: no significant differences |
| | (p = 0.0949; alpha = 0.05) |
| | Left tailed: p = 0.0475; alpha = 0.05 |
| Cerebral White Matter | Mean increase in volume = 2500 mm³ |
| | Two-tailed: p = 0.0.03; alpha = 0.05 |
| | Left-tailed: p = 0.0150; alpha = 0.05 |
| White matter hypointensities | Two-tailed: no significant differences |
| (corrected for cerebral white | (p = 0.0789; alpha = 0.05) |
| matter) | Left tailed: p = 0.9606; alpha = 0.05 |
| | Right-tailed: p = 0.0394; alpha = 0.05 |

The data in Table 3 shows that the increase in white matter volume appears consistent with a hypothesis that ibogaine is triggering neurotrophic repair process.

FIG. 17A is a graph of cerebral white matter volume, in mm³, one month after treatment as a function of cerebral white matter volume, in mm³×10⁵, at baseline (before treatment). FIG. 17B is a graph of the change in cerebral white matter volume, in mm³, taken as the difference in cerebral white matter volume one-month after treatment and at baseline, as a function of cerebral white matter mean volume, in mm³×10⁵, at baseline (before treatment) and one month after treatment. The Bland-Altman plot is often used in test-retest reliability analyses, where the X-axis is the mean of test and re-test; here, the x-axis is the mean of baseline and 1-month post treatment and the Y-axis is the difference between the two. A mean increase in cerebral white matter volume of about 2500 mm³ was observed (two-tailed, p=0.030; alpha=0.05; left-tailed, p=0.0150, alpha=0.05).

The fMRI images were also inspected to determine the difference in volume in regional areas of the brain from baseline to one month after treatment. FIG. 18A is a bar graph showing the effect size of a change in volume in 47 brain regions indicated along the x-axis. Increases in volume were observed in the cerebral white mater (left) and the left ventral DC. Decreases in volume were observed in the posterior corpus callosum and left choroid plexus. The fMRI images were also inspected for whole brain thickness at baseline and one month after treatment. Left hemisphere mean thickness one month after treatment showed no significant difference from baseline (two-tailed, p=0.7146, alpha=0.05); and right hemisphere mean thickness one month after treatment showed no significant difference from baseline (two-tailed, p=09452, alpha=0.05). FIG. 18B is a bar graph showing the effect size of a cortical thickness change for the brain regions indicated along the x-axis, from baseline to one month after treatment. Increases in cortical thickness one-month after treatment was observed in left entorhinal cortex, left-lateral-orbitofrontal pole, parahippocampal gyrus (bilaterally), left insular cortex, and right-pars triangularis (inferior frontal gyrus). The right-pars triangularis (inferior frontal gyrus) acts in response inhibition to suppress actions that are inappropriate in a given context and that interfere with goal-driven behavior. The right and left parahippocampal gyrus are involved in visuospatial processing, including scene perception, navigation, and emotional contextualization. The left orbitofrontal gyrus is involved in impulse control and reward valuation. The left insular cortex is associated with both the affective-perceptual and cognitive-evaluative forms of empathy, as well as awareness of hunger, pain and fatigue. The left entorhinal cortex is a widespread network hub for memory, navigation, and perception of time. The inspection of brain volume and thickness shows no apparent damage to the brain in the treated subjects.

Accordingly, a method for treating a neuropsychiatric disorder is provided. In an embodiment, the neuropsychiatric disorder is TBI. In embodiments, the TBI is mild, moderate or severe. In embodiments, the subject exhibits a persistent symptom caused by the TBI. In an embodiment, the TBI is chronic. In embodiments, the persistent symptom is post-traumatic stress disorder, depression, anxiety and/or suicidal ideation. In other embodiments the persistent symptom is endocrine dysfunction, sleep disturbance, obstructive sleep apnea, chronic pain, orthopedic problems, headache, substance abuse, sexual health problems, cognitive impairment, and/or vestibular or vision impairment.

In another embodiment, a method for treating a neuropsychiatric disorder is provided, where the neuropsychiatric disorder is post-traumatic stress disorder (PTSD). In another embodiment, the neuropsychiatric disorder is obsessive-compulsive disorder. In an embodiment, the subject exhibits a persistent symptom caused by the PTSD or the obsessive-compulsive disorder. In an embodiment, the persistent symptom is depression, anxiety and/or suicidal ideation.

In another embodiment, a method for treating a neuropsychiatric disorder is provided, where the neuropsychiatric disorder is a neurological disorder or a neurodegenerative disease, such as Alzheimer's disease, mild cognitive impairment, Lewy-body dementia, progressive supranuclear palsy, Parkinson's disease, frontotemporal dementia, Tourette's syndrome, suspected chronic traumatic encephalopathy (CTE), multiple sclerosis, stroke, and amyotrophic lateral sclerosis. In another embodiment, the neuropsychiatric disorder is suspected chronic traumatic encephalopathy (CTE) and the administration or treating is effective to slow progression of CTE.

Also provided is a method for treating a neuropsychiatric disorder of the brain in a subject, comprising providing to the subject a cardioprotective agent and a unit dose of an iboga alkaloid or salt thereof, the cardioprotective agent provided in an amount sufficient for a plurality of doses; instructing the subject to self-administer one or more doses of the cardioprotective agent; instructing to administer, or administering, to the subject while under clinical or medical supervision the unit dose of the iboga alkaloid or salt thereof; and optionally administering to the subject while under clinical or medical supervision for administration of the unit dose of ibogaine, a further dose of the cardioprotective agent; and optionally instructing the subject to self-administer one or more doses of the cardioprotective agent subsequent to the administration of the unit dose of the iboga alkaloid or salt thereof.

In another embodiment, a method for treating a neuropsychiatric disorder of the brain in a subject comprises providing a device comprising a plurality of unit doses of a cardioprotective agent and a unit dose of an iboga alkaloid or salt thereof; instructing a subject to self-administer a first portion of the plurality of unit doses of the cardioprotective agent; instructing to administer or administering under clinical or medical supervision the unit dose of the iboga alkaloid or salt thereof to the subject; and administering or instructing the subject to self-administer a second portion of the plurality of unit doses of the cardioprotective agent.

In an embodiment, administering or instructing the subject to self-administer the second portion comprises administering or instructing the subject to self-administer while under clinical or medical supervision for the unit dose of iboga alkaloid or salt thereof. In an embodiment, the method further comprises administering or instructing the subject to self-administer a third portion of the plurality of unit doses of the cardioprotective agent after the second portion of the unit doses of the cardioprotective agent is administered. In an embodiment, the instructing the subject to self-administer comprises instructing the subject to self-administer a unit dose of the cardioprotective agent once daily for a period of 1, 2, 3, 4, 5, 6, 7, 8, 9 or 10 days prior to the unit dose of the iboga alkaloid or salt thereof being administered.

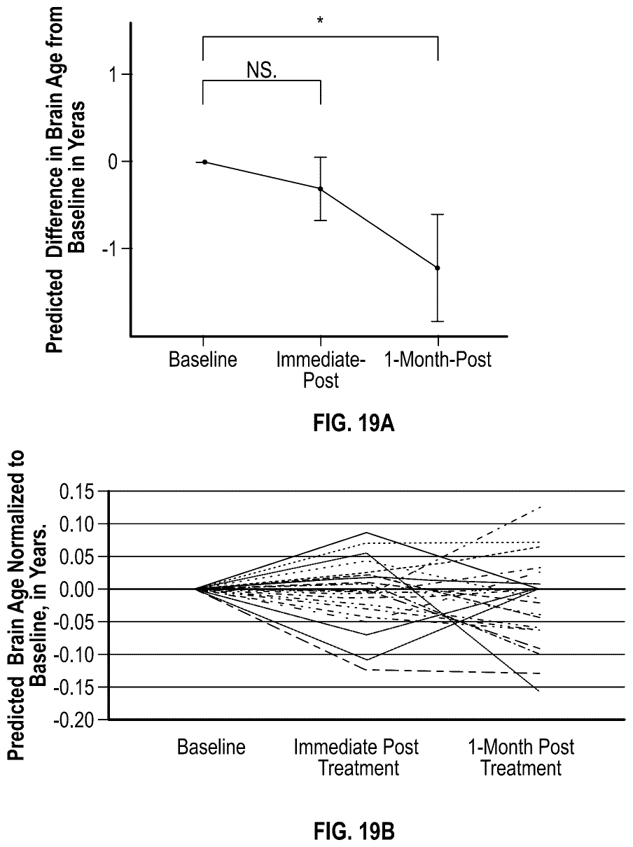

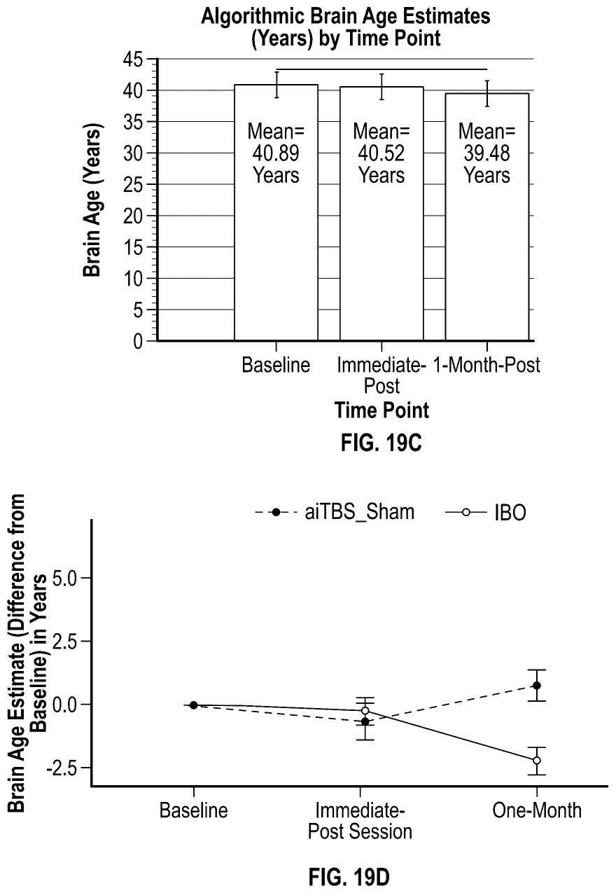

In another aspect, a method for slowing or reversing brain aging in a subject is provided. In an embodiment, the subject is a person a healthy individual or is a person with TBI or with TBI and PTSD or a person at risk of one or both of TBI and PTSD. The method comprises treating the subject with ibogaine, as described herein. Neuroimaging data can be used to predict chronological age in healthy individuals, using machine learning. Neuroimaging-derived age predictions have been explored in the context of different brain diseases (Cole, et al., NeuroImage, 163:115-124 (2017); Cole, et al., Trends in Neurosciences, 40(12):681 (2017))). By training models on healthy individuals, brain-based predictions of age can be made in independent clinical samples. If ‘brain-predicted age’ is greater than an individual's chronological age, this is thought to reflect some aberrant accumulation of age-related changes to the brain (Id.). It has been reported that the long-term effects of traumatic brain injury (TBI) can resemble those observed in normal ageing, suggesting that TBI may accelerate the ageing process (Cole, et al., Annals of Neurology, 77(4):571 (2015)). A predictive model of normal ageing defined using machine learning in healthy individuals, based on magnetic resonance imaging-derived estimates of gray matter and white matter (Id.) was applied to the MRI images from the subjects enrolled in the study of Example 1. FIG. 19A shows the difference in predicted brain age from baseline, in years, for the subjects in the study of Example 1 at baseline, four days after treatment and one month after treatment. There was a significant reversal of brain age from baseline to one month after treatment. The mean decrease in brain age was 1.3 years (corrected p=0.03). FIG. 19B shows the individual brain age trajectories, normalized to baseline, in years, at baseline (v2), 4 days after treatment (v3) and one month after treatment (v4). While the effects of ibogaine on brain aging are complex, a majority of the individual trajectories trend to a decrease in brain age after treatment.

FIG. 19C is a bar graph showing algorithm brain age, in years, for 24 of the subjects in the study of Example 1 prior to ibogaine treatment (baseline), one week after treatment and one month after treatment. At baseline, the brain age estimate for the 24 subjects was 40.89 years. About one week after treatment with ibogaine, the brain age estimate for the 24 subjects was 40.52 years, and one month after treatment, the brain age estimate was 39.48 years. The data is also presented in Tables 4A and 4B. The average decrease in brain age from baseline one month after treatment was 1.4 years. When baseline brain age is contrasted against 1 week post and 1 month post treatment together, there is a significant linear trend for 1 week post and 1 month post to exhibit lower brain ages relative to baseline (Table 4B). When 1 month post treatment is contrasted against baseline and 1 week post treatment, there is a significant linear trend for 1 month post treatment to exhibit lower brain ages relative to both baseline and 1 week post.

TABLE 4A

| | Mean | | | | | | |
|---|---|---|---|---|---|---|---|
| | Difference | | | | | | Cohen's |
| Contrast | (Years) | SE | N | t-stat | p-val | p-adj | d |
| Immediate post (~1 wk) − | −0.3764 | 0.364 | 24 | 1.04 | 0.311 | 0.311 | −0.2114 |
| baseline | | | | | | | |
| 1-mo. post − baseline | −1.408 | 0.468 | 24 | 3.006 | 0.006** | 0.019* | −0.6137 |
| 1 wk post − 1 mo. post | −1.031 | 0.445 | 24 | 2.32 | 0.030* | 0.089 | −0.4729 |

TABLE 4B

| | | | Estimate | 95% | 95% |
|---|---|---|---|---|---|
| Contrast | Weights | Sig | (years) | lower | upper |
| Baseline vs. 1 wk and 1 mo. | 1-.5-.5 | 0.019 | 0.892 | 0.157 | 1.627 |
| Baseline & 1 wk. vs 1 mo. | .5-.5-1 | 0.008 | 1.220 | 0.352 | 2.087 |

Result showing the effect of ibogaine treatment on brain age compared to transcranial magnetic stimulation is shown in FIG. 19D. FIG. 19D is a graph of brain age estimate (difference from baseline), in years, at baseline, and 1-week and 1-month after treatment with ibogaine and a cardioprotective agent (open circles, solid line) and 1-week and 1-month after treatment with accelerated intermittent theta burst stimulation (aiTBS, (solid circles, dashed line). Treatment with an iboga alkaloid provided a reducing in estimated brain age one-month post treatment compared to aiTBS.

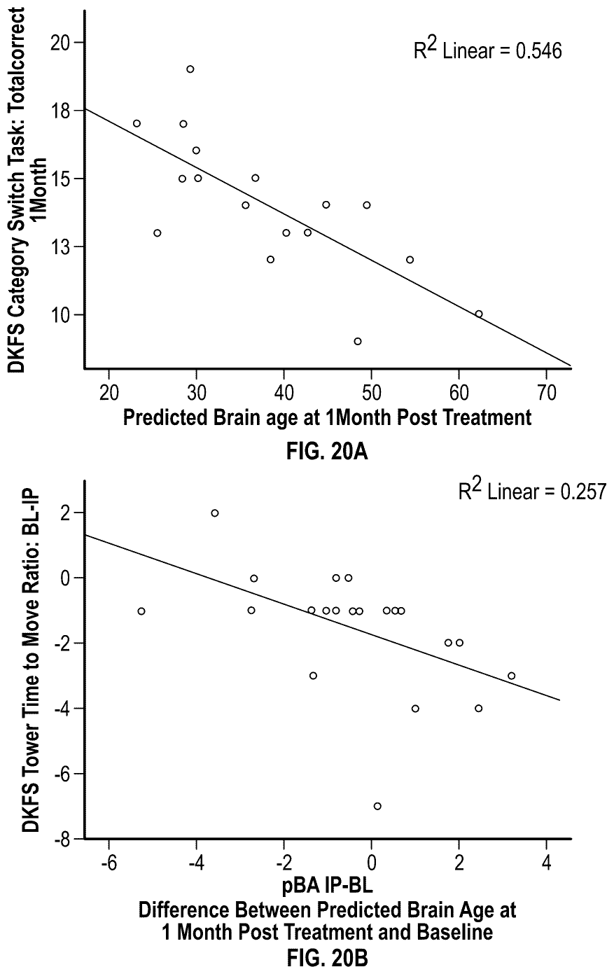

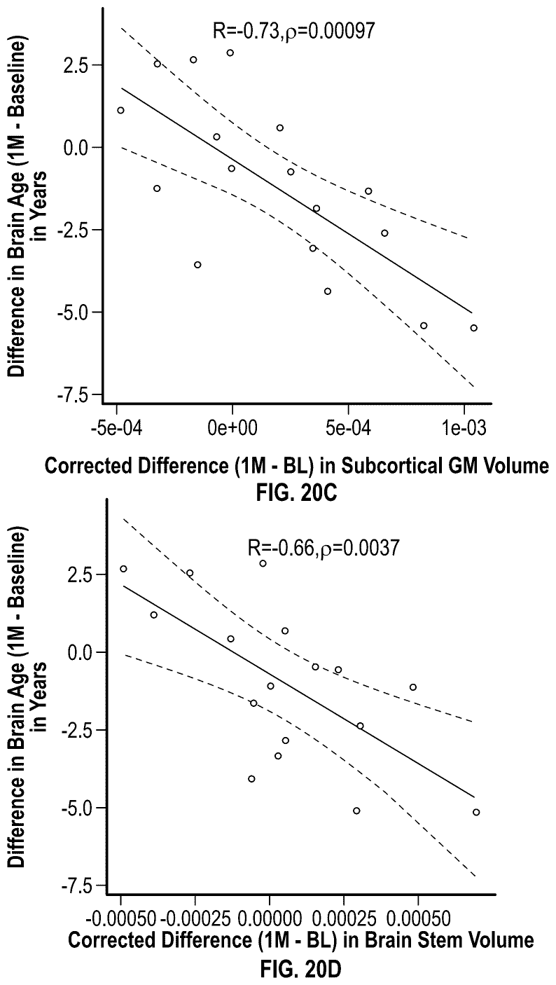

The predicted brain age one month after treatment was predictive of an individual's score on a test of executive function (p=0.001, beta=−0.739), as seen in FIG. 20A. This correlation persisted even when chronological age was controlled for (p=0.008, beta=−0.660). The difference between predicted brain age one month after treatment and baseline was predictive of the difference between time per move on the Delis-Kaplan Executive Function System Tower Test between one month after treatment and baseline, as seen in FIG. 20B. Participants whose brain age became younger from baseline to one month post treatment needed less time on the test from baseline to one month post treatment (p=0.014, beta=−0.507). Controlling for chronological age removed this effect, but it still had a higher beta than chronological age.

FIGS. 20C-20D show the volumetric contributions to brain age change in the subjects treated with ibogaine, where FIG. 20C shows the difference in brain age one month after treatment relative to baseline, in years, in subcortical grey matter volume one month after treatment relative to baseline. The subcortical volume combined the thalamus, caudate, putamen, hippocampus, amygdala, accumbens and ventral DC. FIG. 20D shows the difference in brain age one month after treatment relative to baseline, in years, in brain stem volume one month after treatment relative to baseline.

Accordingly, a method to slow brain aging or to reverse brain aging is contemplated. The method comprises administering to a subject an iboga alkaloid, such as ibogaine, as described herein. In an embodiment, the method further comprises performing an imaging technique on the subject after treatment, to assess effect of the iboga alkaloid on the brain. In an embodiment, the iboga alkaloid is ibogaine. In an embodiment, ibogaine is administered at a dose of between 2-50 mg/kg. In an embodiment, the iboga alkaloid is administered when the subject is under the care of a medical practitioner and/or a psychotherapist. In an embodiment, after a first administration of the iboga alkaloid, the subject rests for 1-7 days and a structural MRI is performed during the rest period or after the rest period. The MRI is analyzed to assess brain age. In an embodiment, the method further comprises administering a cardioprotective agent, before, during and/or after the iboga alkaloid.

In embodiments, a method of treatment to slow or reverse brain aging comprises the steps of performing a screening of the subject to gather medical and/or psychological information; optionally performing a structural MRI and analyzing the MRI with an algorithm to estimate brain age; administering or instructing to administer an iboga alkaloid to the subject; allowing for a period of rest, recovery and/or integration; optionally performing a structural MRI and analyzing the MRI with an algorithm to estimate brain age; and evaluating the subject after the iboga alkaloid treatment. In an embodiment, MRIs before and/or after the iboga alkaloid treatment and analysis with a brain aging algorithm are performed on the subject. In embodiments, the iboga alkaloid is given over a 4-12 hour period and at a dose of 2-50 mg/kg, or at a dose as described herein. In an embodiment, the rest, recovery and/or integration period is from 1-3, 1-4, 1-5, 1-6 or 1-7 days. In an embodiment, the iboga alkaloid is ibogaine. In an embodiment, the method further comprises administering a cardioprotective agent, before, during and/or after the iboga alkaloid.

In an embodiment, the subject to be treated is a healthy subject. In an embodiment, the subject is one at risk of or diagnosed with Alzheimer's disease. In an embodiment, the subject is one at risk of or diagnosed with schizophrenia, multiple sclerosis, alcohol dependence, viral infection, depression, impulsivity or impulse-control disorder, cognitive impairment, excessive sleepiness, chronic low back pain, cigarette smoking, human immunodeficiency virus (HIV), TBI, long COVID-19 and/or obesity. In an embodiment, the subject has mild cognitive impairment. In an embodiment, an imaging technique is used to measure the neuroprotective consequences of ibogaine treatment. In an embodiment, the imaging technique is MRI and the image(s) are used to inform ibogaine dosing or re-dosing.

In an embodiment, brain aging refers to a set of processes which occur in an organism over time, most of which involve the accumulation of diverse and deleterious changes in cells and tissues that increase the likelihood of disease, dysfunction and/or death. Aging changes can be attributed to: (1) developmental and genetic defects, (2) the environment, (3) disease processes, and/or (4) to an inherent process, referred to as the aging process. In an embodiment, aging as used herein refers to a subject's biological age as opposed to chronological age. In an embodiment, the biological age is ascertained by a measure selected from telomere length, grip strength, gum health, lung function, HbA1C level, mean arterial pressure, white blood cell count, cell membrane viscosity, corneal endothelial thickness, cholesterol level, and/or cytomegalovirus optical density. In another embodiment, brain aging is measured with a macroscopic method (e.g., enlarged ventricles, cortical thinning, white matter hyperintensities), a cellular method (e.g. synaptic pruning, axonal loss, mitochondrial changes, alterations to glial cell numbers), a molecular method (altered gene expression, disrupted calcium signaling, epigenetic changes) and/or a behavioral method (cognitive decline, reduced well-being or mood).

MR perfusion techniques measure the quantity of blood that flows through a particular volume of brain tissue, i.e., a voxel, rather than a particular mass. Pseudo-continuous arterial spin labeling (pcASL) MRI is a non-invasive tool to measure cerebral blood flow. Cerebral blood flow is a physiological parameter reflecting the rate of blood supply to the brain and is typically written in units of mL of blood per 100 grams tissue per minute (mL/100 g/min). Cerebral blood flow plays a role in the maintenance of neuronal integrity, and is kept essentially constant in a normal brain over the wide range of systemic blood pressures it encounters. Cerebral blood flow has been shown to be a sensitive marker for cerebrovascular diseases such as stroke and vascular dementia. Changes in perfusion often precede observable structural changes such as atrophy in neurodegenerative diseases.

Arterial spin labeling (ASL) MR was used to measure cerebral blood flow in some of the subjects of the subjects enrolled in the study of Example 1. As described in Example 3, pseudo-continuous ASL (pcASL) was performed on the subject at baseline, about 1 week after treatment, and one-month after treatment. The subjects in Example 3 were treated with ibogaine in combination with a cardioprotective agent as detailed in Example 1. A first metric of interest was change in net quantity (mL/100 g tissue/min) of blood entering different regions of the brain. Images of the left posterior orbital gyrus, left anterior insula, left and right cerebral white matter at baseline and 1-week post treatment (images not shown) were analyzed and compared. Images of the right anterior cingulate gyrus, right middle cingulate gyrus, right superior frontal gyrus medial segment, right posterior orbital gyrus, right cerebral white matter, right and left insula, and right planum polare at baseline and one-month post treatment (images not shown) were analyzed and compared. A paired t-test across 3 time points—baseline (pre-treatment), 1 week post-treatment, and 1 month post-treatment—was employed. No significant changes were seen in absolute cerebral blood flow after treatment with ibogaine (p<0.001).

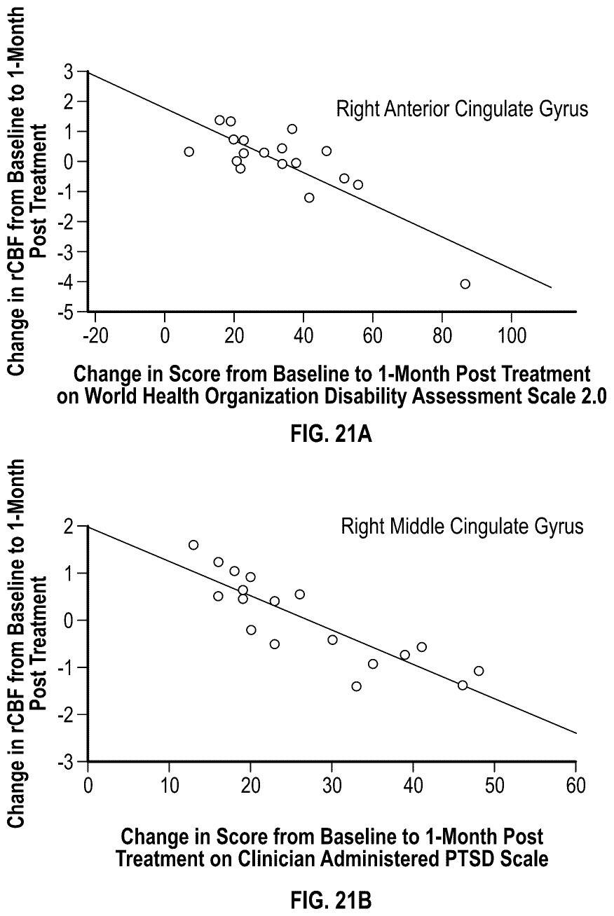

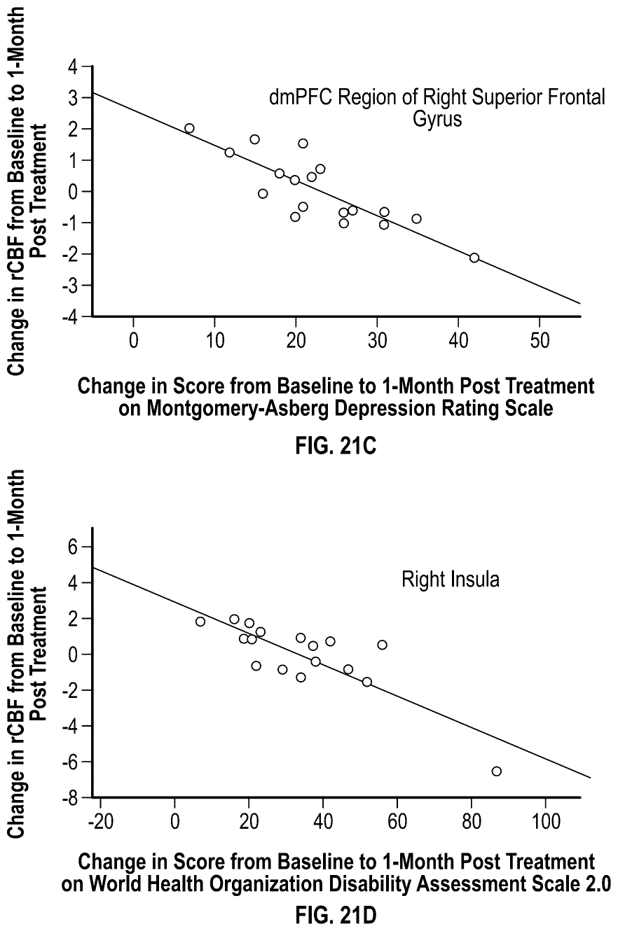

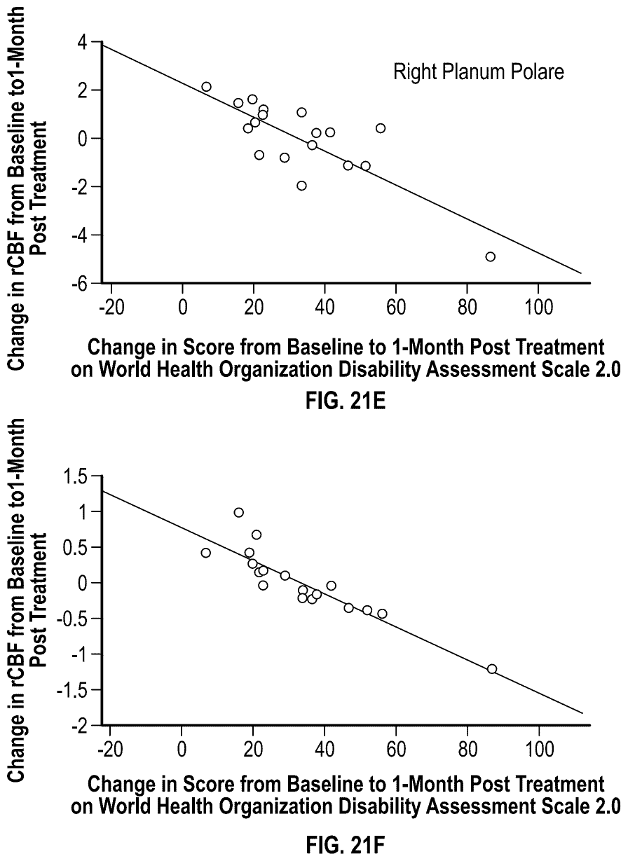

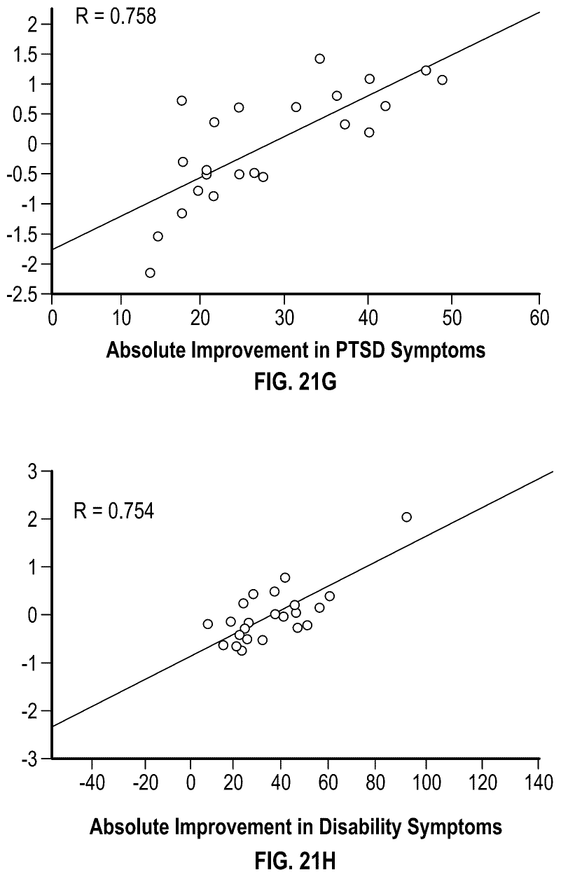

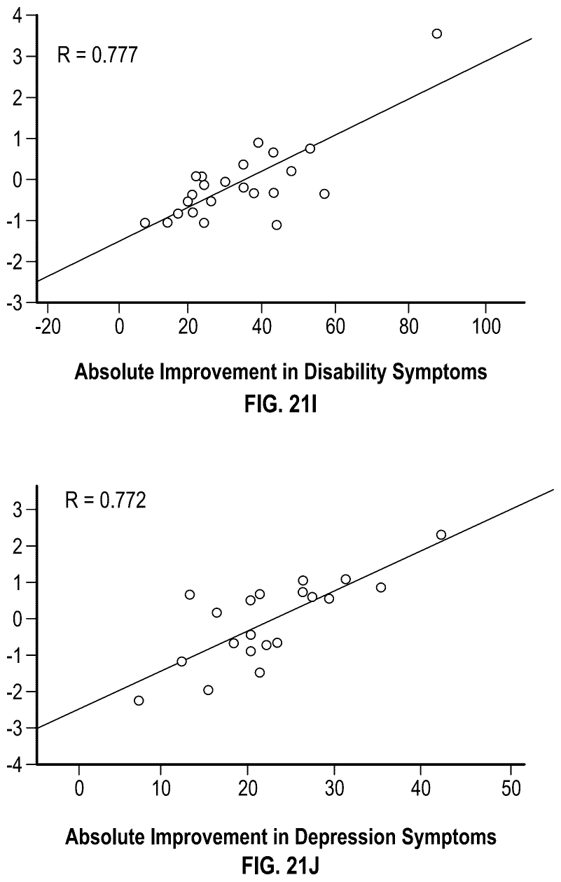

A second metric of interest was regression between change in clinical scores versus change in relative cerebral blood perfusion from baseline to one-month post treatment. Multiple regression with clinical scores at baseline and one-month post treatment was used. Images of the right anterior and middle cingulate gyrus, a component of the limbic system, were obtained (images not shown). The anterior cingulate cortex connects to the emotional limbic system and the cognitive prefrontal cortex. The middle cingulate gyrus is thought to play a role in social behavior and is engaged when monitoring the outcomes of decisions during social interactions. Results are shown in FIGS. 21A-21F.

FIG. 21A shows the correlation between change in relative cerebral blood perfusion in the right anterior cingulate gyrus and change in WHODAS 2.0 score (changes from baseline to one-month post treatment). An increase in relative blood flow in the right anterior cingulate gyrus one month after treatment predicts greater improvement in the disability score.

FIG. 21B is a graph showing the correlation between change in relative cerebral blood perfusion and change in score on the CAPS-5 (a clinician-administered PTSD scale) in the right middle cingulate gyrus, the change from baseline to one-month post treatment. An increase in relative blood flow in the right middle cingulate gyrus one month after treatment predicts greater improvement in the PTSD score.

FIG. 21C is a graph showing the correlation between change in relative cerebral blood perfusion and change in score on the Montgomery-Asberg Depression Rating Scale (MADRS) in the dorsomedial prefrontal cortex (dmPFC) region within the right superior frontal gyrus, the change from baseline to one-month post treatment. The dmPFC plays a role in self-awareness, estimating and executing behavioral responses in a social context. An increase in relative blood flow in a small cluster of the right dmPFC at one month after treatment predicts greater improvement in the depression score.

FIG. 21D shows the correlation between change in relative cerebral blood perfusion in the right insula and change in WHODAS 2.0 score (changes from baseline to one-month post treatment). The insula is associated with the affective-perceptual and cognitive-evaluative forms of empathy. Convergent point of interoceptive processing, or sense of inner self. It may be the cornerstone of our overall awareness. The correlation in FIG. 21D shows that an increase in relative blood flow in the right insula one month after treatment predicts greater improvement in the disability score.

FIG. 21E shows the correlation between change in relative cerebral blood perfusion and change in score on WHODAS 2.0 in the right planum polare, from baseline to one-month post treatment. The right planum polare is a part of the superior temporal gyrus and is involved in auditory processing and receptive language. An increase in relative blood flow in the right planum polare one month after treatment predicts greater improvement in the disability score.

FIG. 21F shows the correlation between change in relative cerebral blood perfusion in the right posterior orbital gyrus and change in WHODAS 2.0 score (changes from baseline to one-month post treatment). The right posterior orbital gyrus receives inputs from the limbic regions of the brain and has a role in processing olfactory and gustatory inputs and in integration of emotions and memories associated with sensory experiences. An increase in relative blood flow in the right posterior orbital gyrus one month after treatment predicts greater improvement in the disability score.

In summary, from the BOLD data, resting state functional connectivity shows reorganization of 22 functional connection pairs in the brain networks within a few days after treatment with ibogaine. This result persisted to the one-month post treatment check in. The ASL data shows relative increases in blood flow post treatment with ibogaine with a change in distribution of blood to different regions of the brain relative to base line blood flow measured prior to ibogaine treatment. Alterations of functional connectivity pairs in the brain network are caused by ibogaine treatment, with a persistent post treatment effect. FIGS. 21G-21J are graphs showing this effect, where the change in relative cerebral blood flow in certain brain regions is correlated with the absolute improvement in the indicated clinical assessment. FIG. 21G shows the change in relative cerebral blood flow in the right middle cingulate gyrus one month after treatment as a function of absolute improvement in PTSD symptoms using the CAPS assessment. FIG. 21H shows the change in relative cerebral blood flow in the dorsomedial prefrontal cortex brain region one month after treatment as a function of absolute improvement in disability symptoms using the WHODAS assessment. FIG. 21I shows the change in relative cerebral blood flow in the right anterior insula one month after treatment as a function of absolute improvement in disability symptoms using the WHODAS assessment. FIG. 21J shows the change in relative cerebral blood flow in the dorsomedial prefrontal cortex brain region one month after treatment as a function of absolute improvement in depression symptoms using the MADRS assessment.

In an embodiment, a method of treating a person at risk of suicide is contemplated. The method comprises administering ibogaine to the person at risk. Ibogaine is administered, in an embodiment, in an amount effective to alter functional connectivity and/or to increase relative cerebral blood flow in one or more of the limbic region, the DMPFC region, and the insula. In an embodiment, the limbic region comprises the cingulate gyrus. In an embodiment, ibogaine is administered orally as ibogaine hydrochloride. In an embodiment, ibogaine is administered at a dose of 13-50 mg/kg. In an embodiment, ibogaine is administered in 3-4 doses within a 2 hour period. In an embodiment, fMRI or ASL is performed on the person before and/or after treatment to determine presence or extent of increase in cerebral blood flow relative to cerebral blood flow before treatment with ibogaine. In an embodiment, the person at risk of suicide is a person with at least one prior suicide attempt.

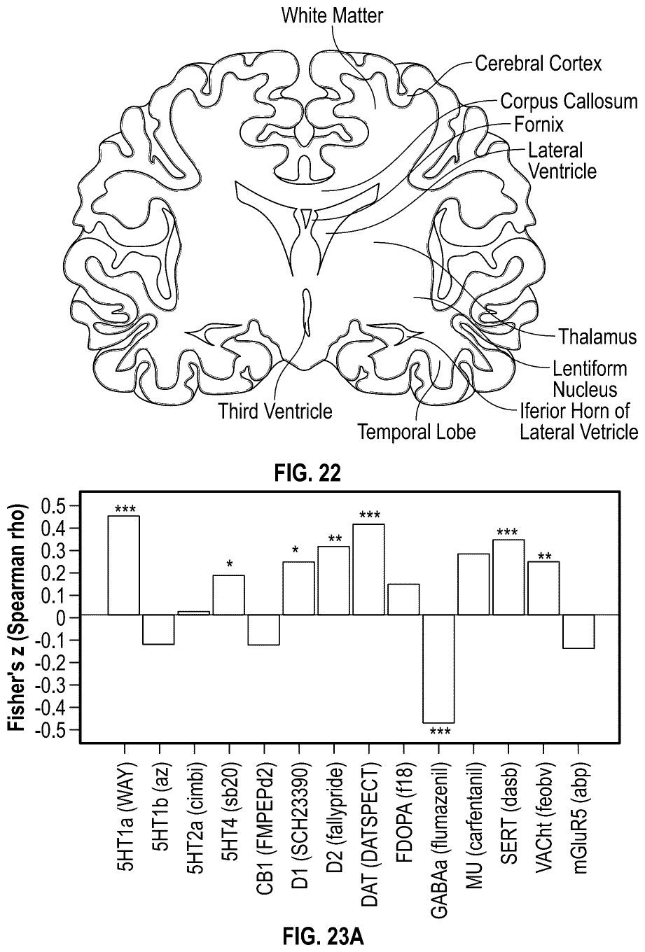

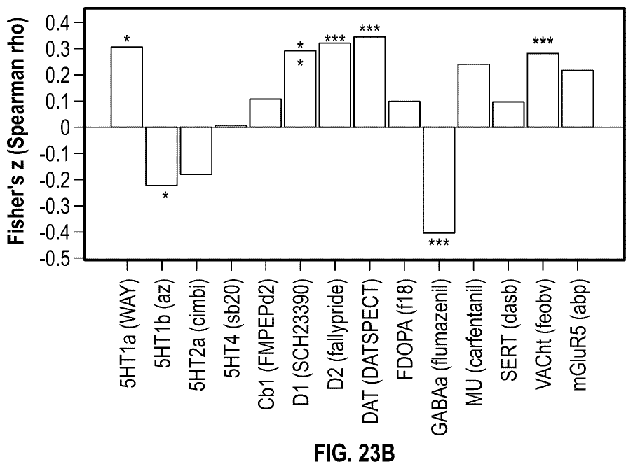

White matter lies beneath the gray matter cortex in the brain. FIG. 22 is a cross-sectional diagram of the brain showing the white matter and other regions of interest. White matter is composed of millions of bundles of axons (nerve fibers) that connect neurons in different brain regions into functional circuits. A correlation between perfusion disturbances and extent of white matter disease has been reported, where subjects with later-stage white matter lesions exhibit decreased cerebral blood flow in both white and gray matter (Bastos-Leite, et al., Am. J Neuroradiol, 29(7):1296-1301 (2008)). The relative changes in cerebral blood perfusion from baseline to one month post treatment with ibogaine and a cardioprotective agent receptors mapped with positron emission tomography (PET) was evaluated (Dukart, J. et al., Hum. Brain Mapp., 42:555-556 (2021)). The receptors included 5-HT1a (serotonin 5-hydroxytryptamine receptor subtype 1a), 5-HT1b (5-HT subtype 1b), 5-HT2a (5-HT subtype 2a), 5-HT4 (5-HT subtype 4), CB1 (cannabinoid receptor 1), D1 (dopamine D1), D2 (dopamine D2), DAT (dopamine transporter), F-DOPA (dopamine synthesis capacity), GABAa (gamma-aminobutric acid), Mu receptor, SERT (serotonin trans-porter), vesicular acetylcholie transporter (VAChT), and mGluR5 (metabotropic glutamate receptor subtype 5). FIG. 23A is a bar graph showing the relative cerebral brain perfusion change from baseline to four days after treatment, using Fisher's Z (Spearman rho), for the indicated PET map receptor. Also indicated on the x-axis is the PET tracer used for the receptor map (Dukart et al, supra). FIG. 23B is a similar graph for change in relative cerebral brain perfusion from baseline to one-month after treatment.

Stroke is a common cause of disability. Ischemia is characterized by too little blood to supply an adequate amount of oxygen and nutrients to a part of the brain. Ischemia can be due to thrombosis, embolism or systemic hypoperfusion. A majority of strokes are due to ischemic cerebral infarction. A person's prognosis following stroke or an ischemic event is based on the size, location and volume of the stroke area, the person's age, and medical comorbidities. A community-based study in the US evaluated 220 ischemic stroke survivors of age 65 years or older and identified the following neurologic deficiencies at 6 months after stroke: hemiparesis (50%), cognitive deficits (46%), hemianopia (20%), aphasia (19%0, and sensory deficits (15%). The observed disability measures 6 months after stroke included depression symptoms (35%), unable to walk unassisted (31%), social disability (30%), institutionalization (26%), bladder incontinence (22%). The proportion of patients at 6-12 months after stroke who had returned to paid employment was just over 50% (Kelly-Hayes, J. et al., J. Stroke Cerebrovasc Dis., 12(3):119-126 (2003); de Haan, R. J. et al., Stroke, 26(3):402-408 (1995)). A current treatment paradigm for ischemic stroke is to identify stroke, attempt to restore blood perfusion, generally with a 3-4.5 hour window for intravenous thrombolysis or mechanical thrombectomy. Then, medical stabilization, workup to treat causes and initiation of secondary prevention measures. Next, inpatient rehabilitation starts about one week post stroke and may continue for 2-6 weeks or more depending on stroke severity. No large randomized clinical trials have demonstrated efficacy for inpatient rehabilitation therapy. In the chronic phase corresponding to greater than or equal to six months post stroke, where physical therapy and occupational therapy are rarely offered, rehabilitation therapy shows efficacy in some large trials. After stroke, the plasticity process is initiated in an attempt to compensate for both the lesion itself and its remote effects. Changed neuronal activity and connectivity in terms of function and structure can be detected in perilesional and remote regions and even in the contralateral hemisphere, which are assumed to be the mechanisms underlying spontaneous recovery. Generally, increased neural activity and connectivity in the ipsilesional hemisphere were reported as indicators or better functional recovery. Attempts to promote plasticity have led to disappointing results thus far (Su, F. et al., Front Neurol., 11:554089 (2020); Dimyan, M. et al., Nat. Rev. Neurol., 7:76-85 (2011); Crofts, A. et al., J. Neuroimaging, 30:5-14 (2020)).

A method for treating ischemic stroke and/or for improving recovery from stroke comprising administering ibogaine is contemplated. In an embodiment, the method comprises administering ibogaine to a person who has had a stroke and has been medically stabilized. In an embodiment, the person is treated with ibogaine while in a monitored setting, such as the hospital or an inpatient rehabilitation clinic or facility. Subsequent to ibogaine treatment, rehabilitation therapy is initiated, such as physical therapy, occupational therapy, speech or other cognitive therapy. Recovery of the person is monitored, and if a subsequent period of brain plasticity is needed for further rehabilitation therapy, a further ibogaine treatment is given. In an embodiment, treatment with ibogaine is preceded by or is concurrent with treating the subject with a cardioprotective agent, as described herein.

Repeated mild traumatic brain injuries can contribute to a condition termed chronic traumatic encephalopathy (CTE). There are four stages of CTE—Stage I characterized by headaches and loss of concentration and attention; Stage II characterized by depression, explosivity, and short-term memory loss; State III characterized by executive dysfunction and cognitive impairment; and Stage IV characterized by dementia, word finding difficulty and aggression. The symptoms of CTE are progressive and include cognitive deficits such as memory and executive functioning deficits; behavior and personality changes, such as aggression, paranoia and impulsivity; changes in mood, such as depression, anxiety and suicidality; and Parkinsonism and other speech and gait abnormalities. CTE has been described in professional sport setting, combat-related injuries, and after motor vehicle accidents. It likely encompasses what has been referred to as dementia pugilistica, a late-emerging dementia in boxers. One convenience sample of deceased football (NFL) players found CTE in 110 of the 111 sampled.

In CTE, neuropathology is consistent with a tauopathy, with an anatomic distribution involving superficial cortical layers that is distinct from that seen in Alzheimer's disease. Pathognomonic lesion is an accumulation of abnormal hyperphosphorylated tau (p-tau) in neurons and astroglia distributed around small blood vessels at the depths of cortical sulci and in an irregular pattern. Apolipoprotein E (APOE) genotype may be a risk factor for CTE as it is for Alzheimer's disease. APOE genotype was associated with severity of neurologic deficits among high- but not low-exposure boxers. APOE genotype was associated with severity of neurologic deficits among high- but not low-exposure boxers. Neuroimaging in aging NFL players shows a greater burden of white matter lesions and regional blood flow differences corresponded to regions associated with impaired neurocognitive performance (Acta Neuropathol., 131(1):75 (2016); JAMA, 278(2):136 (1997); Neurosurgery, 47(3):651 (2000); JAMA Neurol., 70(3):326 (2013)).

In an embodiment, a method for treating a person at risk of CTE is provided. The method comprises administering ibogaine to the person prior to, during or subsequent to an activity or action that may cause a TBI episode. For example, a soldier, an athlete, and/or a person with a history of TBI and/or concussions can be treated prior to an activity that can be TBI and/or concussion inducing, with ibogaine. In an embodiment, the method reduces risk of development of CTE in a person at risk thereof. In an embodiment, treatment with ibogaine is preceded by or is concurrent with treating the subject with a cardioprotective agent, as described herein.

### IV. Examples

The following examples are illustrative in nature and are in no way intended to be limiting.

### Example 1

### Safety and Efficacy of Ibogaine Treatment in Special Operations Veterans

Thirty, male, navy seal or army special forces veterans, ages 33-58 years (mean 44.5 years), were enrolled. Of the 30 participants, 27 were Caucasian, 1 was Hispanic and 2 were Native American. Body mass index was between 17-35 kg/m². Baseline characteristics of the subjects is set forth in Table 1-1.

TABLE 1-1
Baseline characteristics of the subjects enrolled in study

| | Number of subjects enrolled |
|---|---|
| Characteristic | with characteristic |
| TBI (by history) | mTBI: 28 |
| | moderate TBI: 1 |
| | moderately severe TBI: 1 |
| TBI with loss of consciousness (LOC) | 28 |
| (by history) | |
| PTSD Lifetime (per MINI¹) | Full diagnosis: 25 |
| | Subclinical: 4 |
| | No PTSD: 1 |
| Major depressive disorder (MDD) | Current: 15 |
| Lifetime (Per MINI) | Past: 6 |
| | No MDD: 9 |
| Substance use disorder (SUD) (per MINI) | None: 15 |
| | Alcohol use disorder: 10 |
| | Alcohol and SUD: 5 |
| History of suicidal ideation | None: 12 |
| (per SCID², MINI) | With suicidal ideation: 11 |
| History of suicide attempts | 7 |
| Combat exposure (score per CES³) | Range: 17-37 |
| | 27/30 had score 25+ |

¹MINI = Mini International Neuropsychiatric Interview
²SCID = structured clinical interview for DSM Disorders
³CES = Combat Exposure Scale

Enrolled subjects were assessed using clinical intake, diagnostic and psychological evaluations (Table 1-1), subjective reporting of symptoms, MRI, EEG and blood draw. In particular, following enrollment, participants undertook initial baseline evaluations over a secure video platform with a clinical neuropsychologist between two months and one week prior to in-person assessments, including review of medical and psychiatric history, history of combat exposures, history of TBI and blast exposure, and a psychodiagnostic interview. All participants presented with a history of TBI, according to the Ohio State University Screening for TBI exposure and the DoD TBI classification (Maas, A. I. R. et al., Lancet Neurol. 21, 1004-1060 (2022)). In addition, to quantify blast exposure, the Boston Assessment of TBI—Lifetime was administered (Administration, U. D. of V. A., Veterans Health. VA.gov|Veterans Affairs).

To prepare for treatment with ibogaine, a medication washout period was designed for each subject and expectation and intentions were discussed. Two to three days prior to treatment, each subject underwent neuropsychological testing, mood measures, structural and functional imaging and EEG.

With subjects in the fasting state, an intravenous infusion of 1 gram of magnesium sulfate was administered 1-2 hours prior to treatment. The oral ibogaine dosing protocol consisted of an initial test dose of 2-3 mg/kg of ibogaine hydrochloride (98+% pure). Depending on response, after about 40 minutes additional doses of ibogaine up to a total of <14 mg/kg were administered within a total 2-hour period. Approximately 12 hours after administration of ibogaine, participants were administered another intravenous infusion of 1 gram magnesium sulfate. Medical staff (MD, RN, or EMT) were onsite at a ratio of at least 1 staff per 2 patients throughout treatment for monitoring and management, but no specific coaching or psychological support was provided during treatment. For 12-16 hours following ibogaine administration, blood pressure and pulse oximetry were monitored three times a day, and QTc was monitored via continuous 5-lead ECG.

Post treatment, each subject was assessed four days after treatment, one month after treatment, and, if willing, one year after treatment. The post-treatment assessments conducted at four days and at one month after treatment included neuropsychological testing, measures of PTSD, depression, anxiety, mystical experiences, functional imaging and EEG. Post-treatment assessments conducted at one year after treatment included PTSD, depression, and anxiety measures.

Statistical analysis of the data used a p-value to ascertain the likelihood that the differences seen do not reflect a true difference, where a p-value below 5% (0.05) was considered significant. Effect size was measured using Cohen's D, and effects larger than 0.8 were considered strong. When testing for changes in multiple factors, the more the factors the more potential for erroneous inferences. A statistical method for correction of multiple tests was implemented.

World Health Organization Disability Assessment Scale 2.0 (WHODAS 2.0) was used as a measure of function. WHODAS was used to assess the impact of health conditions across six life domains (cognition, mobility, self-care, interpersonal, life activities, and community participation) and it is sensitive to change over time (Gold, L. H., J. Am. Acad. Psychiatry Law 42, 9 (2014). Each item in the assessment is rated on a scale ranging fromno problems to extreme problems (Üstün, T. B. et al., Measuring health and disability: Manual for WHO disability assessment schedule WHODAS 2.0. (World Health Organization, 2010)). To capture inter-individual variability in disability the WHODAS complex scoring method was used. Raw scores were converted to a metric ranging from 0 (no disability) to 100 (full disability), by calculating the ratio of the participant's score relative to the maximum possible score in each domain as well as to the total score (Id.). A score of 20%-39% was considered mild, 40%-59% was moderate, 60%-79% was moderate-severe and 80%-100% was severe. Scores from baseline were compared to scores from one-month post treatment.

Cognitive assessments were done to determine the effect of ibogaine treatment on various cognitive domains including executive functions, memory, processing speed, attention, and working memory.

Assessments for psychological symptoms were conducted to determine the effect of treatment on clinician-rated depressive, anxious, and post-traumatic symptoms.

Imaging and EEG were performed to determine the effect of treatment on brain functional connectivity, structure, blood flow/perfusion and electrical activity.

Functional magnetic resonance imaging (fMRI) was used as a biomarker to assess whole brain connectivity, default mode and task positive network (DMN-TPN) cross-talk and connectivity with PCC as seed. Superior rostral anterior cingulate cortex (srACC) increased coupling with para-hippocampal, hippocampal, amygdala, and medial temporal lobe (MTL) regions.

Neuropsychological testing of the enrolled subjects was designed to examine abilities over several domains of cognitive functioning, including estimated intelligence quotient (IQ), verbal fluency, working memory (visual and auditory), long term memory (visual and auditory), processing speed, sustained attention, problem solving, ability to perceive patterns, executive functioning, such as ability to hold and manipulate or update information in the head, inhibition, ability to organize, plan, and develop solutions to problems. In addition, assessments examining cognitive abilities over several domains of functioning were done to evaluate the safety of ibogaine use. The neuropsychological tests included WASI-II (Weschler Abbreviated Scale Intelligence) for estimated IQ (verbal comprehension and perceptual reasoning), WAIS-IV (Wechsler Adult Intelligence Scale) for working memory and processing speed (digit span, arithmetic symbol search, coding), D-KEFS (Delis-Kaplan Executive Function System) for executive functioning (trail making test, verbal fluency, color-word inhibition, tower test), HVLT (Hopkins Verbal Learning Test) and BVMT-R (Brief Visuospatial Memory Test) for learning and memory, and CPT-3 (Conner's Continuous Performance Test) for attention.

Domains of cognition included language (verbal fluency, receptive language), working memory (hold and use small amounts of temporarily stored information, time taken to compete tasks), executive functioning (cognitive switching, flexibility, multitasking), organizing, planning and finding solutions, inhibition, filtering distractions), learning and memory (ability to learn, store and retrieve auditory and visual information), attention (ability to filter distractions and remain focused).

Self-reporting measures by enrolled subjects included PTSD symptoms, mood, hopelessness, sleep, pain, function, moral injury, mystical experiences, personality (baseline only), childhood trauma (baseline only) and combat experiences (baseline only).

Clinician administered scales included CAPS-5 (Clinician-administered PTSD Scale for DSM-5), which is a gold standard assessment of PTSD symptom severity; MADRS (Montgomery-Asberg Depression Rating Scale), a measure for depressive symptom severity; and Hamilton-Anxiety Rating Scale (HAM-A) (Shear, M. K. et al., Depress. Anxiety 13, 166-178 (2001)) a measure for anxiety symptom severity; SCID-5 (Structured Clinical Interview for DSM-5) Overview for a historical and environmental context of symptom presentation; MINI DSM-5 (Mini International Neuropsychiatric Interview from DSM-5) for a structured psychodiagnostics assessment; BAT-L (Boston Assessment of TBI—Lifetime) for a lifetime history of TBI and blast exposure; and Ohio State University TBI Identification Method (Short Form) for a lifetime history of TBI. MADRS testing provides an indication of depressive symptoms, CAPS-5 provides an indication of PTSD and SIGH-A provides an indication of anxiety. Collectively these overlap to provide indication of mood, concentration, sleep and tension.

For the CAPS-5, the test version with a 30-item structured interview of PTSD symptoms over the past week was used, using a 0 (“Absent”) to 4 (“Extreme/Incapacitating”) scale, with possible total scores ranging from 0 to 80. The score range 23 to 34 is considered moderate PTSD while a higher score represents severe PTSD (Weathers, F. W. et al., Psychol. Assess. 30, 383-395 (2018)).

The MADRS is a clinician-administered 10-item scale assessing the severity of depression symptoms. Items are rated on a scale of 0 (no abnormality) to 6 (severe) (Montgomery, S. A. et al., Br. J. Psychiatry J. Ment. Sci. 134, 382-389 (1979)). A total score of 0-6 indicates no depression, 7-19 mild depression, 20-34 moderate depression, 35-59 severe depression, and 60+ very severe depressive symptoms (Miller, M. J., J. Affect. Disord. 77, 255-260 (2003)).

To assess anxiety symptoms, the Hamilton Anxiety Rating Scale was used. This scale includes 14-items assessing both psychic and physical symptoms of anxiety. Items are rated on a scale from 0 (no symptoms) to 4 (severe symptoms). Total score ranges were: no or minimal anxiety (<7), mild (8-14), moderate (15-23), and severe anxiety symptoms (>24) (Matza et al., Int. J Methods Psychiatr. Res. 19, 223-232 (2010)).

Results are shown in Table 1, Table 1-2, Table 1-3, Table 1-4, Table 1-5, Table 1-6 and FIGS. 1-8.

TABLE 1-2
WHODAS 2.0 Subscales.

| | | | | | | One- | | | |
|---|---|---|---|---|---|---|---|---|---|
| | | | Baseline vs | | | Month | Baseline vs | | |
| WHODAS | | Post- | Post-Treatment | | | (Mean ± | One-Month | | |
| 2.0 Subscale | Baseline | Treatment | F(1, 75) | p(FDR) | D | SD) | F(1, 75) | p(FDR) | D |
| Cognition | 36.1% ± | 19.2% ± | 40.38 | <0.001 | 0.96 | 5.0% ± | 94.84 | <0.001 | 2.38 |
| | 14.6% | 18.7% | | | | 7.9% | | | |
| Community | 35.6% ± | 24.7% ± | 8.24 | 0.006 | 0.53 | 5.9% ± | 50.15 | <0.001 | 1.78 |
| Participation | 21.1% | 22.7% | | | | 13.1% | | | |
| Life | 41.1% ± | 31.6% ± | 8.25 | 0.006 | 0.48 | 9.1% ± | 57.02 | <0.001 | 1.44 |
| Activities | 24.8% | 26.8% | | | | 16.2% | | | |
| Interpersonal | 32.3% ± | 21.7% ± | 13.34 | 0.001 | 0.60 | 4.8% ± | 63.07 | <0.001 | 1.63 |
| | 20.4 | 19.6% | | | | 9.5% | | | |
| Self Care | 5.2% ± | 1.9% ± | 5.01 | 0.028 | 0.37 | 1.0% ± | 7.21 | 0.009 | 0.51 |
| | 9.2% | 4.7% | | | | 2.9% | | | |
| Mobility | 14.7% ± | 7.2% ± | 11.33 | 0.001 | 0.53 | 1.3% ± | 22.06 | <0.001 | 0.83 |
| | 15.7% | 13.5% | | | | 3.3% | | | |

TABLE 1-3
Sensitivity analyses including only participants meeting relevant diagnostic criteria according to the MINI at baseline.
For suicidal ideation, analysis included only participants with non-zero suicidal ideation at baseline.

| | | | Baseline vs 1-wk. post | | | | | | Baseline vs 1-mo. post | | | | |
|---|---|---|---|---|---|---|---|---|---|---|---|---|---|
| | | 1-wk | Included | | | | | 1-mo. | Included | | | | |
| | Baseline | post | N | F | df | p(FDR) | D | post | N | F | df | p(FDR) | D |
| CAPS- | 35.7 ± | 4.4 ± | 23 | 221.16 | (1, 57) | <0.001 | 2.75 | 6.0 ± | 23 | 197.84 | (1, 57) | <0.001 | 2.98 |
| 5 | 11.0 | 5.2 | | | | | | 8.7 | | | | | |
| MADRS | 31.3 ± | 3.6 ± | 15 | 217.62 | (1, 36) | <0.001 | 4.11 | 6.4 ± | 15 | 178.49 | (1, 36) | <0.001 | 3.15 |
| | 6.5 | 3.6 | | | | | | 7.7 | | | | | |
| HAM- | 23.8 ± | 4.2 ± | 14 | 100.36 | (1, 36) | <0.001 | 2.52 | 5.1 ± | 14 | 100.36 | (1, 36) | <0.001 | 2.33 |
| A | 7.6 | 3.4 | | | | | | 6.3 | | | | | |
| Suicidal | % | % | Included | X² | — | p(FDR) | — | % | Included | X² | — | p(FDR) | — |
| Ideation | Reporting | Reporting | | | | | | Reporting | | | | | |
| (MADRS | 100% | 0% | 14 | 28.00 | — | <0.001 | — | 14% | 14 | 21.00 | — | <0.001 | — |
| Q10)¹ | | | | | | | | | | | | | |

% Reduction vs

| | | |
|---|---|---|
| Baseline | Response Rate | Remission Rate |

| | 1-wk. | 1-mo. | 1-wk. | 1-mo. | 1-wk. | 1-mo. |
|---|---|---|---|---|---|---|
| | post | post | post | post | post | post |
| CAPS- | 87% ± | 86% ± | 96% | 100% | 83% | 83% |
| 5 | 17% | 19% | | | | |
| MADRS | 89% ± | 81% ± | 100% | 93% | 73% | 67% |
| | 11% | 21% | | | | |
| HAM- | 82% ± | 80% ± | 100% | 86% | 86% | 71% |
| A | 14% | 23% | | | | |

TABLE 1-4
Sensitivity analyses including only participants with mild TBI

| | | | | | | | | | Baseline vs | | | | |
|---|---|---|---|---|---|---|---|---|---|---|---|---|---|
| | | | Baseline vs 1-wk post | | | | | | 1-mo. post | | | | |
| | | 1-wk. | Included | | | | | 1-mo. | Included | | | | |
| | Baseline | post | N | F | df | p(FDR) | D | post | N | F | df | p(FDR) | D |
| CAPS- | 31.7 ± | 3.6 ± | 28 | 187.23 | (1, 72) | <0.001 | 2.28 | 4.8 ± | 28 | 170.80 | (1, 72) | <0.001 | 2.49 |
| 5 | 13.0 | 4.5 | | | | | | 8.1 | | | | | |
| MADRS | 25.2 ± | 2.7 ± | 28 | 211.50 | (1, 72) | <0.001 | 2.55 | 3.6 ± | 28 | 196.12 | (1, 72) | <0.001 | 2.70 |
| | 8.7 | 3.2 | | | | | | 5.9 | | | | | |
| HAM- | 20.7 ± | 3.3 ± | 28 | 145.63 | (1, 72) | <0.001 | 2.02 | 3.7 ± | 28 | 144.31 | (1, 72) | <0.001 | 2.07 |
| A | 8.6 | 3.3 | | | | | | 4.6 | | | | | |
| Suicidal | % | % | Included | X² | — | p(FDR) | — | % | Included | X² | — | p(FDR) | — |
| Ideation | Reporting | Reporting | N | | | | | Reporting | N | | | | |
| (MADR | SI | SI | | | | | | SI | | | | | |
| Q10) | 46% | 0% | 28 | 16.93 | — | <0.001 | — | 7% | 28 | 11.02 | — | <0.001 | — |

% Reduction vs

| | | |
|---|---|---|
| Baseline | Response Rate | Remission Rate |

| | 1-wk. | 1-mo. | 1-wk. | 1-mo. | 1-wk. | 1-mo. |
|---|---|---|---|---|---|---|
| | post | post | post | post | post | post |
| CAPS- | 89% ± | 88% ± | 96% | 100% | 89% | 85% |
| 5 | 14% | 17% | | | | |
| MADRS | 87% ± | 87% ± | 100% | 96% | 85% | 85% |
| | 24% | 17% | | | | |
| HAM- | 82% ± | 82% ± | 96% | 93% | 89% | 85% |
| A | 19% | 21% | | | | |

TABLE 1-5
Statistical results (t, df, and FDR-corrected p-values) associated
with main effects of time point for each LME model

| All participants included | t | df | P(FDR) |
|---|---|---|---|
| WHODAS Total | 9.25 | 76 | <0.001 |
| WHODAS Cognition | 9.62 | 76 | <0.001 |
| WHODAS Community Participation | 6.99 | 76 | <0.001 |
| WHODAS Life Activities | 7.38 | 76 | <0.001 |
| WHODAS Interpersonal | 7.91 | 76 | <0.001 |
| WHODAS Self Care | 2.71 | 76 | 0.009 |
| WHODAS Mobility | 4.70 | 76 | <0.001 |
| CAPS-5 | 9.06 | 79 | <0.001 |
| MADRS | 9.65 | 79 | <0.001 |
| HAM-A | 9.25 | 79 | <0.001 |
| Including only participants meeting | | | |
| relevant diagnostic criteria | | | |
| CAPS-5 | 8.47 | 58 | <0.001 |
| MADRS | 6.93 | 37 | <0.001 |
| HAM-A | 6.07 | 40 | <0.001 |
| Including only participants with mild TBI | | | |
| CAPS-5 | 8.56 | 73 | <0.001 |
| MADRS | 9.30 | 73 | <0.001 |
| HAM-A | 8.94 | 73 | <0.001 |

TABLE 1-6
Neuropsychological tests statistical results (t,
df, and FDR-corrected p-values) associated with
main effects of time point for each LME model.

| Neuropsychological Construct | T | Df | P(FDR) |
|---|---|---|---|
| Detection | 4.50 | 64 | <0.001 |
| Reaction time | 3.02 | 64 | 0.008 |
| Sustained attention | 0.59 | 64 | 0.589 |
| Verbal memory | 2.52 | 76 | 0.024 |
| Visuospatial memory | 2.08 | 77 | 0.052 |
| Processing speed | 6.39 | 77 | <0.001 |
| Cognitive inhibition | 3.77 | 77 | 0.001 |
| Cognitive flexibility composite | 4.20 | 77 | <0.001 |
| Phonemic fluency | 4.70 | 77 | <0.001 |
| Working memory | 2.44 | 76 | 0.027 |
| Problem solving | 2.51 | 76 | 0.024 |
| Semantic fluency | 1.35 | 77 | 0.215 |

FIG. 1A is a bar graph showing combined results for D-KEFS testing of inhibition score for the enrolled subjects at baseline, at immediate (4 days) post treatment, and one month post treatment. Four days after treatment, an increase in inhibition score was observed, with a p-value of <0.001 and an effect size of 1.22 relative to baseline. One month after treatment, an increase in inhibition score was observed, with a p-value of 0.001 and an effect size of 0.62, both relative to baseline. FIGS. 1B-1C are graphs showing individual results of D-KEFS testing of cognitive inhibition. From the individual results, no indication of an adverse effect on processing speed was observed.

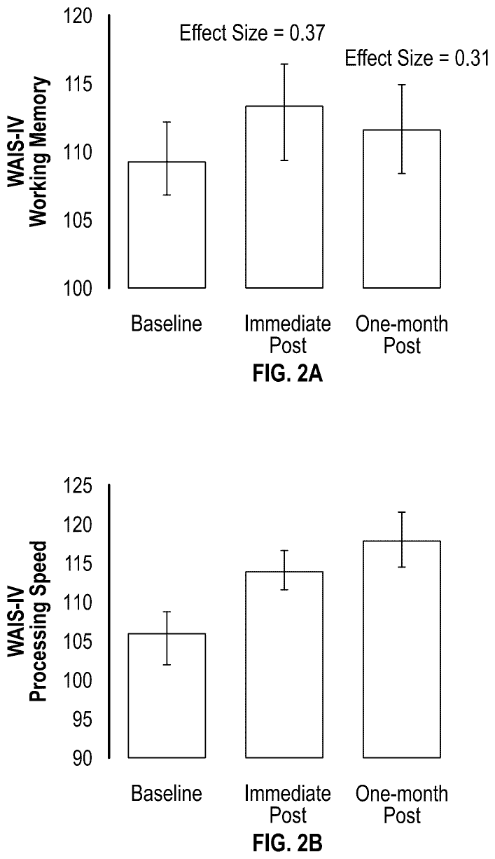

FIGS. 2A-2B are bar graphs showing results from assessment of working memory (FIG. 2A) and processing speed (FIG. 2B) using the Working Memory Index (WAIS-IV) test at baseline, at immediate (4 days) post treatment, and one month post treatment. Four days after treatment, an increase in working memory score was observed (FIG. 2A), with an effect size of 0.37 relative to baseline. The improved working memory score was observed one month after treatment, with an effect size of 0.31 relative to baseline. Four days after treatment, an increase in processing speed score was observed (FIG. 2B), with p-value of less than 0.001 and an effect size of 0.97, both relative to baseline. The improved processing speed score continued to increase one month after treatment, with p-value of less than 0.001 and an effect size of 1.34, both relative to baseline.

FIGS. 3A-3B are graphs showing PTSD symptom severity as measured using Clinician-Administered PTSD Scale for DSM-5 (CAPS-5), collectively for the enrolled subjects (FIG. 3A) and individually (FIG. 3B), at baseline, at immediate (4 days) post treatment, and one month post treatment. The CAPS-5 scale is a gold standard in PTSD assessment and included a 30-item structured interview to assess PTSD symptoms over the prior week. The score ranges are mild (12-22), moderate (23-34) and severe (above 35). Four days after (“immediate post”) treatment, a decrease in PTSD severity score was observed (FIG. 3A and Table 1-3 in Example 1), with an effect size of 2.75 and a p-value of 0.001, both relative to baseline, for the enrolled subjects. The reduced PTSD severity score was maintained, as seen by the score one month after treatment, with an effect size of 2.98 relative to baseline and a p-value of less than 0.001 relative to baseline. On average, participants experienced a reduction of 88% in symptom severity at one month follow-up relative to baseline (prior to treatment) (FIG. 3B). One month following treatment, 80% of the subjects were lower than mild symptom severity (FIG. 3B).

FIGS. 4A-4B are graphs showing depression symptom severity as measured using Montgomery-Asberg Depression Rating Scale (MADRS), collectively for the enrolled subjects (FIG. 4A) and individually (FIG. 4B), at baseline, immediately after (4 days) treatment, and one month post treatment. MADRS measures depressive symptom severity, and score ranges are mild (7-19), moderate (20-34) and severe (above 34). Four days after (“immediate post”) treatment, depression symptom severity score decreased from a baseline score of about 26 to about 3, an effect size of 4.11 and a p-value of 0.001, both relative to baseline, for the enrolled subjects (FIG. 4A and Table 1-3 in Example 1). The reduced depression symptom severity score was maintained, as seen by the score one month after treatment, with an effect size of 3.15 relative to baseline and a p-value of less than 0.001 relative to baseline. Individual results for the enrolled subjects are shown in FIG. 4B, where 82% of the subjects meet the criteria for remission with a MADRS score of less than 7. 96% of the subjects met the criteria for response, with a reduction of more than 50% in symptoms severity.

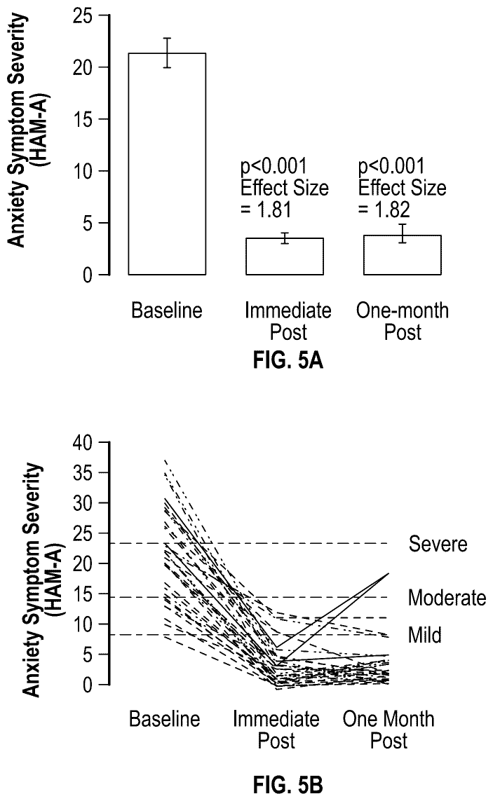

FIGS. 5A-5B are graphs showing anxiety symptom severity as measured using Hamilton-Anxiety Rating Scale (HAM-A), collectively for the enrolled subjects (FIG. 5A) and individually (FIG. 5B), at baseline, immediately after (4 days) treatment, and one month post treatment. HAM-A is a structured interview to quantify anxiety symptom severity, and score ranges are mild (8-14), moderate (15-23) and severe (above 24). Four days after (“immediate post”) treatment, anxiety symptom severity score decreased from a baseline score of about 21 to about 7, an effect size of 1.81 and a p-value of 0.001, both relative to baseline, for the enrolled subjects (FIG. 5A and Table 1-3 in Example 1). The reduced anxiety symptom severity score was maintained, as seen by the score one month post treatment, with an effect size of 1.82 relative to baseline and a p-value of less than 0.001 relative to baseline. Individual results for the enrolled subjects are shown in FIG. 5B, where one month post treatment 90% of the subjects scored below the mild anxiety range, and 93% of the subjects showed a clinically meaningful reduction of anxiety symptoms (e.g., a more than 50% reduction in anxiety symptoms) (FIG. 5B).

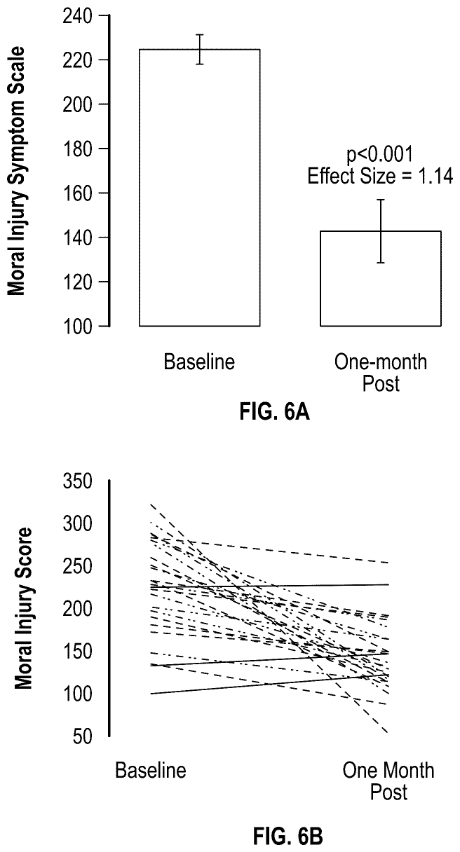

A Moral Injury Symptom Scale (MISS) was also used to assess the subjects before and one month after treatment. Moral injury is when one acts in a manner or witnesses behaviors that go against an individual's values and moral beliefs. This may include betrayal by leadership or peers with adverse outcomes. Symptoms include guilt, remorse, shame, disgust, anger, inability to forgive oneself, engaging in self-sabotaging behaviors, such as feeling undeserving, affecting work and relationships, and questioning spirituality. The MISS is a multidimensional measure to assess the severity of moral injury symptoms. Possible score range is 45-450, with higher scores indicating more severe moral injury (Koenig et al., J Relig Health, 57:249-265 (2018)). Results are shown in FIGS. 6A-6B.

FIGS. 6A-6B are graphs showing results from the MISS assessment, collectively for the enrolled subjects (FIG. 6A) and individually (FIG. 6B), at baseline and one month post treatment. Prior to treatment, subjects had an average moral injury symptoms score of about 220 (FIG. 6A). One month after treatment, the average score for the treated subjects was about 140, with an effect size of 1.14 and a p-value of 0.001 (FIG. 6A). 88% of the subjects shows a reducing in moral injury score, as seen by the individual results in FIG. 6B.

A measure of disability for health and disability on life functions is the World Health Organization Disability Assessment Schedule 2.0 (WHODAS). The items are designed to be sensitive to change due to a treatment, and the measure assesses disability over the past 30 days. The items measured are cognition, self-care, mobility, getting along, life activity, and participation in society. The WHODAS measure gives a rating of 1 for no disability, 2 for mild disability and 3 for moderate disability. Individual and collective results for the subjects are shown in FIGS. 7A-7B. FIG. 7A is a graph showing results from the World Health Organization Disability Assessment Scale 2.0 (WHODAS 2.0) assessment for each subject at baseline (prior to treatment), about one week after treatment, and one month after treatment. The dashed line represents the means of the individual scores. FIG. 7B is a bar graph of self-reported disability using the WHODAS total scores at baseline and one-month after treatment. The baseline self-reported disability WHODAS total score of about 2.5 decreased significantly (p<0.001) in the subjects after treatment, as seen by the score of just over 1 in the one-month post treatment assessment.

Alcohol consumption in the enrolled subjects was also evaluated. At baseline, 11 of the 30 enrolled subjects reported drinking at least four days per week and 12 of the 30 enrolled subjects reported drinking at least four days per week over the last month. Some of the subjects reported drinking daily. One month following treatment, none (zero) of the enrolled subjects reported drinking more than three times per week. 28 of the 30 enrolled subjects reported drinking less than twice per week over the past month.

Intoxication by alcohol was also evaluated in the enrolled subjects. At baseline, 9 of the 30 enrolled subjects reported being intoxicated more than once over the last 30 days, with some subjects reporting being intoxicated for 25 of the last 30 days. One month following treatment, one participant reported four episodes of intoxication after reporting 23 episodes at baseline.

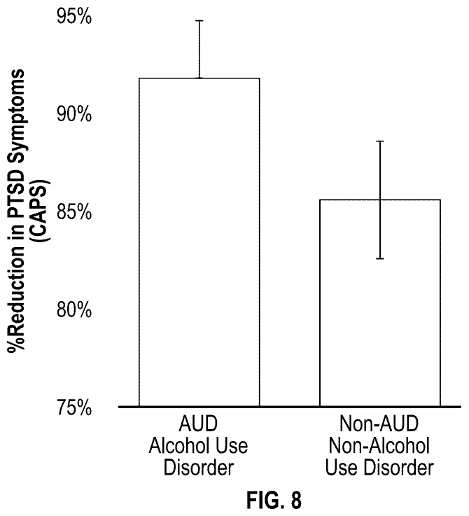

The data in FIG. 3B for PTSD symptom severity was used to evaluate whether alcohol use is related to treatment response. Of the enrolled subjects, 37% at baseline reported alcohol use disorder and 63% reported a non-alcohol use disorder. FIG. 8 is a bar graph of the percent reduction in PTSD symptoms from baseline to one-month post treatment assessed using CAPS-5 for the subjects reporting an alcohol use disorder and a non-alcohol use disorder. The subjects with a non-alcohol use disorder reported a larger percent reduction compared to the subjects with an alcohol use disorder, however both patient populations benefited from treatment with ibogaine and a cardioprotective agent. Reducing alcohol use is a possible mechanism by which ibogaine alleviates PTSD.

### Example 2

### Functional MRI Imaging as a Biomarker

The subjects enrolled in the study described in Example 1 were evaluated by functional MRI at baseline, one week after treating with ibogaine and one month after treating with ibogaine, as described in Example 1. The subjects were asked to clear their mind of any particular thoughts in order to capture the brain at rest or in its ‘default’ state. An a priori region of interest was determined for analysis and 5000 functional connectivity pairs were tested. The underlying alteration in brain patterns (connectivity) due to an intervention was evaluated. The functional connectivity networks evaluated are listed in Table 2-1.

TABLE 2-1
Functional connectivity networks

| Functional | | |
|---|---|---|
| Network | Description | Abbreviation |
| Central | Executive function and goal oriented, | CEN |
| executive | cognitively demanding tasks, working | |
| | memory, decision making | |
| Dorsal | Goal-directed, voluntary control of | DAN |
| attention | visuospatial attention | |
| Default mode | Self-referential processing and the | DMN |
| | so-called ‘stream of consciousness,” | |
| | introspective mental imagery, self- | |
| | reflection and self-awareness | |
| Limbic | Processing emotion and memory, | LN |
| | including the hippocampus, the | |
| | amygdala and the hypothalamus | |
| Somatomotor | Sensor motor integration | SMN |
| Salience | Modulating the switch between DMN | SN |
| | and CEN (attention) | |
| Visual | Visual perception and processing | VN |

Results are shown in FIGS. 10A-10C, FIG. 11, FIGS. 12A-12B, FIGS. 13A-13C, FIG. 14, FIG. 15, FIGS. 16A-16N.

### Example 3

### Cerebral Blood Flow

Arterial spin labeling (ASL) MRW was used to measure cerebral blood flow. Eighteen subjects of the subjects enrolled in the study of Example 1, all male veterans with a history of TBI, were evaluated with pseudo-continuous ASL (pcASL) at baseline, 1-2 days after treatment with ibogaine (orally administered), and one-month after treatment with ibogaine, in combination with a cardioprotective agent as described in Example 1.

A first metric of interest was change in net quantity (mL/100 g tissue/min) of blood entering different regions of the brain. Images of the left posterior orbital gyrus, left anterior insula, left and right cerebral white matter at baseline and 1-2 days post treatment (images not shown) were analyzed and compared. Images of the right anterior cingulate gyrus, right middle cingulate gyrus, right superior frontal gyrus medial segment, right posterior orbital gyrus, right cerebral white matter, right and left insula, and right planum polare at baseline and one-month post treatment (images not shown) were analyzed and compared.

A paired t-test across 3 time points—baseline (pre-treatment), 1-2 days post-treatment, and one month post-treatment—was employed. No significant changes were seen in absolute cerebral blood flow after treatment with ibogaine (p<0.001).

A second metric of interest was regression between change in clinical scores versus change in relative cerebral blood perfusion from baseline to one-month post treatment. Multiple regression with clinical scores at baseline and one-month post treatment was used. Images of the right anterior and middle cingulate gyrus, a component of the limbic system, were obtained (not shown). Results showing correlation of clinical scores from some of the assessments described in Example 1 with change in cerebral blood flow from baseline to one-month post treatment for certain brain regions are shown in FIGS. 21A-21F.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A composition, comprising:
an orally administrable dosage form comprising an iboga alkaloid or salt thereof and a salt of magnesium, potassium or calcium, the salt of magnesium, potassium or calcium present in the dosage form in an amount effective to achieve a physiologic effect to reduce risk of a drug-induced long QT syndrome.

2. The composition of claim 1, wherein the dosage form is a solid.

3. The composition of claim 2, wherein the solid dosage form is for oral ingestion.

4. The composition of claim 2, wherein the solid dosage form is selected from the group consisting of a sublingual composition, a buccal composition, a powder, a capsule, a pill, and a tablet.

5. The composition of claim 1, wherein the salt of magnesium, potassium or calcium is formulated for immediate release upon oral administration of the dosage form.

6. The composition of claim 1, wherein the iboga alkaloid or salt thereof is formulated for delayed release after oral administration of the dosage form.

7. The composition of claim 6, wherein iboga alkaloid or salt thereof is formulated for release at least two hours after the salt of magnesium, potassium or calcium is released from the dosage form.

8. The composition of claim 1, wherein the dosage form is a liquid.

9. The composition of claim 8, wherein the liquid dosage form is selected from the group consisting of an elixir, a syrup, a suspension and a solution.

10. The composition of claim 1, comprising a salt form of magnesium selected from the group consisting of magnesium aspartate, magnesium aspartate hydrochloride, magnesium bisglycenate, magnesium carbonate, magnesium chloride, magnesium citrate, magnesium gluconate, magnesium glycinate, magnesium hydroxide, magnesium lactate, magnesium malate, magnesium oxide, magnesium sulfate, and magnesium taurate.

11. The composition of claim 10, wherein the dosage form comprises between about 50-8000 mg of a magnesium salt.

12. The composition of claim 1, comprising a salt form of potassium selected from the group consisting of potassium chloride, potassium citrate, and potassium bicarbonate.

13. The composition of claim 1, comprising a salt form of calcium selected from the group consisting of calcium gluconate, calcium chloride, calcium citrate, calcium glubionate, calcium gluceptate, calcium carbonate, and calcium acetate.

14. The composition of claim 1, wherein the iboga alkaloid or salt thereof is ibogaine or an analog of ibogaine.

15. The composition of claim 12, wherein the iboga alkaloid or salt thereof is ibogaine hydrochloride.

16. The composition of claim 1, wherein the iboga alkaloid or salt thereof is a salt of ibogaine present in the dosage from in an amount between about 200-2500 mg.

17. The composition of claim 1, wherein the iboga alkaloid or salt thereof is ibogaine hydrochloride and the salt of magnesium, potassium or calcium is magnesium sulfate.

18. A kit, comprising:
a device comprising a plurality of unit doses of a cardioprotective agent and a unit dose of an iboga alkaloid or salt thereof; and
instructions for use.

19. A method for treating a neuropsychiatric disorder of the brain, comprising:
administering, or instructing to administer, to a subject a cardioprotective agent in an amount effective to achieve a physiologic effect to reduce risk of long QT syndrome;
administering an iboga alkaloid or salt thereof.

20. A method for treating a neuropsychiatric disorder of the brain in a subject, comprising:
providing to the subject a cardioprotective agent and a unit dose of an iboga alkaloid or salt thereof, the cardioprotective agent provided in an amount sufficient for a plurality of doses;
instructing the subject to self-administer one or more doses of the cardioprotective agent;
instructing to administer, or administering, to the subject while under clinical or medical supervision the unit dose of the iboga alkaloid or salt thereof; and
optionally administering to the subject while under clinical or medical supervision for administration of the unit dose of ibogaine, a further dose of the cardioprotective agent; and
optionally instructing the subject to self-administer one or more doses of the cardioprotective agent subsequent to the administration of the unit dose of the iboga alkaloid or salt thereof.
